(19) United States
(12) Patent Application Publication
(10) Pub. No.: US 2026/0090841 A1
(43) Pub. Date: Apr. 2, 2026

(54) SYSTEMS AND METHODS FOR HEMORRHAGE IDENTIFICATION AND DETERMINATION OF FUNCTIONAL OUTCOME AND TREATMENT

(71) Applicant: The Cleveland Clinic Foundation, Cleveland, OH (US)

(72) Inventors: Ahmed Kashkoush, Shaker Heights, OH (US); Mark Bain, Cleveland Heights, OH (US)

(21) Appl. No.: 19/342,833

(22) Filed: Sep. 29, 2025

Related U.S. Application Data
(60) Provisional application No. 63/699,961, filed on Sep. 27, 2024.

(51) Int. Cl. A61B 34/10; A61B 6/03; A61B 6/50; G06T 7/00; G06T 7/11; G06T 7/136; G06T 7/73; G06V 10/70; G16H 50/20

(52) U.S. Cl. CPC A61B 34/10; A61B 6/032; A61B 6/501; A61B 6/504; G06T 7/0014; G06T 7/11; G06T 7/136; G06T 7/73; G06V 10/70; G16H 50/20; G06T 2207/10081; G06T 2207/20081; G06T 2207/30016; G06V 2201/031

(57) ABSTRACT

One or more computing devices, systems and/or methods are provided. In some examples, one or more images of a brain of a patient may be received. One or more anatomic landmarks in the one or more images may be determined. A hemorrhage region in the one or more images may be identified. A normalized hemorrhage map indicative of the hemorrhage region may be generated based upon the one or more anatomic landmarks and the hemorrhage region in the one or more images. A spatial representation of the hemorrhage region may be generated based upon a comparison of the normalized hemorrhage map to a dissimilarity heat map. A treatment plan for the patient may be determined based upon the spatial representation of the hemorrhage region.

### RELATED APPLICATION

This application claims priority to U.S. Provisional Patent Application No. 63/699,961, filed on Sep. 27, 2024, entitled “AN AUTOMATED METHOD FOR IMAGE SEGMENTATION, STEREOTACTIC LOCALIZATION, AND FUNCTIONAL OUTCOME PREDICTION OF BASAL GANGLIA HEMORRHAGES,” which is incorporated herein by reference in its entirety.

### BACKGROUND

Intracranial hemorrhage is a significant medical problem associated with disability and sometimes mortality. Quickly identifying a hemorrhage region and/or diagnosing hemorrhage expansion may lead to improved outcomes, such as by enabling a patient to be treated more quickly and/or effectively in a critical period associated with the condition.

### SUMMARY

In accordance with the present disclosure, one or more computing devices, systems and/or methods are provided. In some examples, one or more images of a brain of a patient may be received. One or more anatomic landmarks in the one or more images may be determined. A hemorrhage region in the one or more images may be identified. A normalized hemorrhage map indicative of the hemorrhage region may be generated based upon the one or more anatomic landmarks and the hemorrhage region in the one or more images. A spatial representation of the hemorrhage region may be generated based upon a comparison of the normalized hemorrhage map to a dissimilarity heat map. A treatment plan for the patient may be determined based upon the spatial representation of the hemorrhage region.

In some examples, a computed tomography (CT) scan of a brain of a patient may be received. A hemorrhage region in the CT scan may be identified. A brain tissue region, in the CT scan, excluding the hemorrhage region may be identified. A normalized CT-density associated with the hemorrhage region may be determined based upon the hemorrhage region and the brain tissue region. A probability of hemorrhage expansion may be determined based upon the normalized CT-density associated with the hemorrhage region.

In some examples, a CT scan of a brain of a patient may be received. One or more anatomic landmarks in the CT scan may be identified. A hemorrhage region in the CT scan may be identified. A normalized hemorrhage map indicative of the hemorrhage region may be generated based upon the one or more anatomic landmarks and/or the hemorrhage region in the CT scan. A spatial representation of the hemorrhage region may be generated based upon a comparison of the normalized hemorrhage map to a dissimilarity heat map. A brain tissue region, in the CT scan, excluding the hemorrhage region may be identified. A normalized CT-density associated with the hemorrhage region may be determined based upon the brain tissue region and the hemorrhage region in at least one of the CT scan or the normalized hemorrhage map. A treatment plan for the patient may be generated based upon the spatial representation of the hemorrhage region and the normalized CT-density. In some examples, one or more treatments may be performed based upon the treatment plan.

### DESCRIPTION OF THE DRAWINGS

The patent or application file contains at least one drawing executed in color. Copies of this patent or patent application publication with color drawing(s) will be provided by the Office upon request and payment of the necessary fee.

While the techniques presented herein may be embodied in alternative forms, the particular embodiments illustrated in the drawings are only a few examples that are supplemental of the description provided herein. These embodiments are not to be interpreted in a limiting manner, such as limiting the claims appended hereto.

FIG. 1 is an illustration of a scenario involving various examples of networks that may connect servers and clients.

FIG. 2 is an illustration of a scenario involving an example configuration of a server that may utilize and/or implement at least a portion of the techniques presented herein.

FIG. 3 is an illustration of a scenario involving an example configuration of a client that may utilize and/or implement at least a portion of the techniques presented herein.

FIG. 4 is a flow chart illustrating an example method, in accordance with some embodiments.

FIG. 5A is a component block diagram illustrating a system of evaluating an image of a brain of a patient and/or determining a functional outcome and/or a treatment plan for the patient, in accordance with some embodiments.

FIG. 5B is a component block diagram illustrating identification of anatomical landmarks in a system of evaluating an image of a brain of a patient and/or determining a functional outcome and/or a treatment plan for the patient, in accordance with some embodiments.

FIG. 5C is a component block diagram illustrating identification of a hemorrhage region in a system of evaluating an image of a brain of a patient and/or determining a functional outcome and/or a treatment plan for the patient, in accordance with some embodiments.

FIG. 5D is a component block diagram illustrating generation of a normalized hemorrhage region map in a system of evaluating an image of a brain of a patient and/or determining a functional outcome and/or a treatment plan for the patient, in accordance with some embodiments.

FIG. 5E is a component block diagram illustrating determination of a stereotactic reference frame in a system of evaluating an image of a brain of a patient and/or determining a functional outcome and/or a treatment plan for the patient, in accordance with some embodiments.

FIG. 5F is a component block diagram illustrating generation of a dissimilarity heat map in a system of evaluating an image of a brain of a patient and/or determining a functional outcome and/or a treatment plan for the patient, in accordance with some embodiments.

FIG. 5G is a component block diagram illustrating generation of a spatial representation of a hemorrhage region in a system of evaluating an image of a brain of a patient and/or determining a functional outcome and/or a treatment plan for the patient, in accordance with some embodiments.

FIG. 6 is a flow chart illustrating an example method, in accordance with some embodiments.

FIG. 7A is a component block diagram illustrating a system of evaluating an image of a brain of a patient and/or determining a hemorrhage expansion probability and/or a treatment plan for the patient, in accordance with some embodiments.

FIG. 7B is a component block diagram illustrating generation of a hemorrhage region representation in a system of evaluating an image of a brain of a patient and/or determining a hemorrhage expansion probability and/or a treatment plan for the patient, in accordance with some embodiments.

FIG. 8 is a flow chart illustrating an example method, in accordance with some embodiments.

FIG. 9 is an illustration of a scenario featuring an example non-transitory machine readable medium in accordance with one or more of the provisions set forth herein.

### DETAILED DESCRIPTION

Subject matter will now be described more fully hereinafter with reference to the accompanying drawings, which form a part hereof, and which show, by way of illustration, specific example embodiments. This description is not intended as an extensive or detailed discussion of known concepts. Details that are known generally to those of ordinary skill in the relevant art may have been omitted, or may be handled in summary fashion.

The following subject matter may be embodied in a variety of different forms, such as methods, devices, components, and/or systems. Accordingly, this subject matter is not intended to be construed as limited to any example embodiments set forth herein. Rather, example embodiments are provided merely to be illustrative. Such embodiments may, for example, take the form of hardware, software, firmware, medicine, clothing design, or any combination thereof.

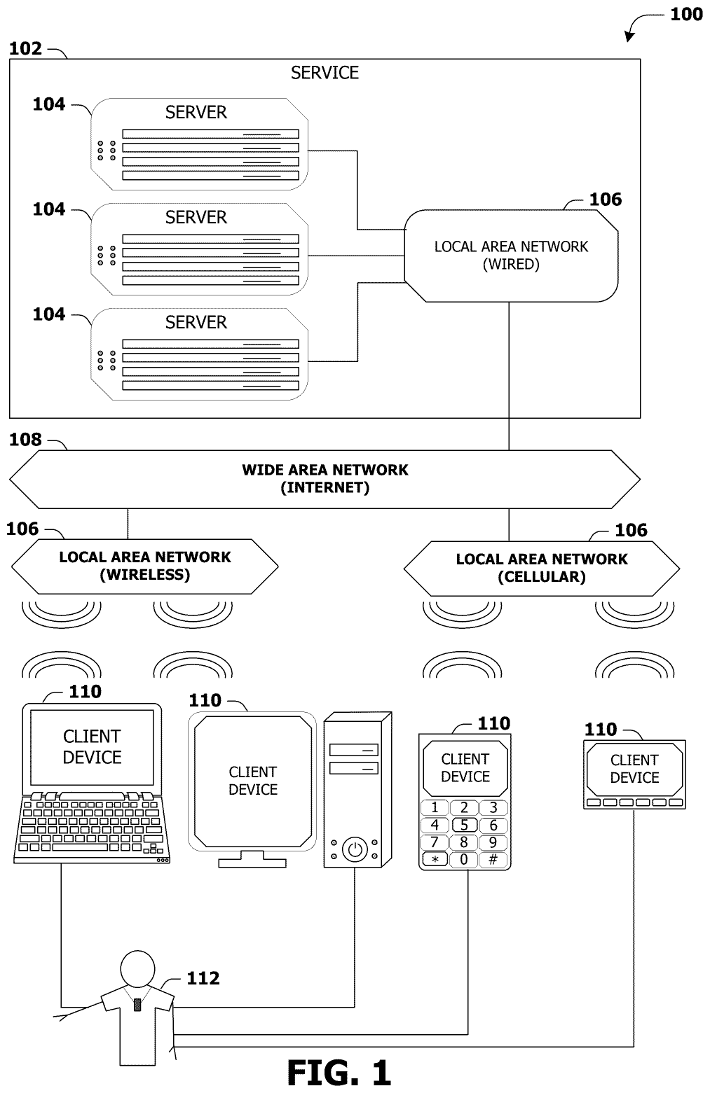

FIG. 1 is an interaction diagram of a scenario 100 illustrating a service 102 provided by a set of servers 104 to a set of client devices 110 via various types of networks. The servers 104 and/or client devices 110 may be capable of transmitting, receiving, processing, and/or storing many types of signals, such as in memory as physical memory states.

In the scenario 100 of FIG. 1, the service 102 may be accessed via a wide area network 108 (WAN) by a user 112 of one or more client devices 110, such as a portable media player (e.g., an electronic text reader, an audio device, or a portable gaming, exercise, or navigation device); a portable communication device (e.g., a camera, a phone, a wearable or a text chatting device); a workstation; and/or a laptop form factor computer. The respective client devices 110 may communicate with the service 102 via various connections to the wide area network 108.

One or more client devices 110 may comprise a cellular communicator and may communicate with the service 102 by connecting to the wide area network 108 via a wireless local area network 106 (LAN) provided by a cellular provider.

Alternatively and/or additionally, one or more client devices 110 may communicate with the service 102 by connecting to the wide area network 108 via a wireless local area network 106 provided by a location such as the user's home or workplace. The wireless local area network 106 may, for example, be a WiFi (Institute of Electrical and Electronics Engineers (IEEE) Standard 802.11) network or a Bluetooth (IEEE Standard 802.15.1) personal area network.

It may be appreciated that the servers 104 and the client devices 110 may communicate over various types of networks. Exemplary types of networks that may be accessed by the servers 104 and/or client devices 110 include mass storage, such as network attached storage (NAS), a storage area network (SAN), or other forms of computer or machine readable media.

The servers 104 of the service 102 may be interconnected directly, or through one or more other networking devices, such as routers, switches, and/or repeaters. The servers 104 may utilize a variety of physical networking protocols, such as Ethernet and/or Fiber Channel, and/or logical networking protocols, such as variants of an Internet Protocol (IP), a Transmission Control Protocol (TCP), and/or a User Datagram Protocol (UDP).

The servers 104 of the service 102 may be internally connected via a local area network 106. The local area network 106 may be organized according to one or more network architectures, such as server/client, peer-to-peer, and/or mesh architectures, and/or a variety of roles, such as administrative servers, authentication servers, security monitor servers, data stores for objects such as files and databases, business logic servers, time synchronization servers, and/or front-end servers providing a user-facing interface for the service 102.

The local area network 106 may be a wired network where network adapters on the respective servers 104 are interconnected via cables (e.g., coaxial and/or fiber optic cabling), and may be connected in various topologies (e.g., buses, token rings, meshes, and/or trees). The local area network 106 may include, e.g., analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as may be known to those skilled in the art.

Alternatively and/or additionally, the local area network 106 may comprise one or more sub-networks, such as may employ differing architectures, may be compliant or compatible with differing protocols and/or may interoperate within the local area network 106. Additionally, a variety of local area networks 106 may be interconnected; e.g., a router may provide a link between otherwise separate and independent local area networks 106.

In the scenario 100 of FIG. 1, the local area network 106 of the service 102 is connected to a wide area network 108 that allows the service 102 to exchange data with other services 102 and/or client devices 110. The wide area network 108 may encompass various combinations of devices with varying levels of distribution and exposure, such as a public wide-area network (e.g., the Internet) and/or a private network (e.g., a virtual private network (VPN) of a distributed enterprise).

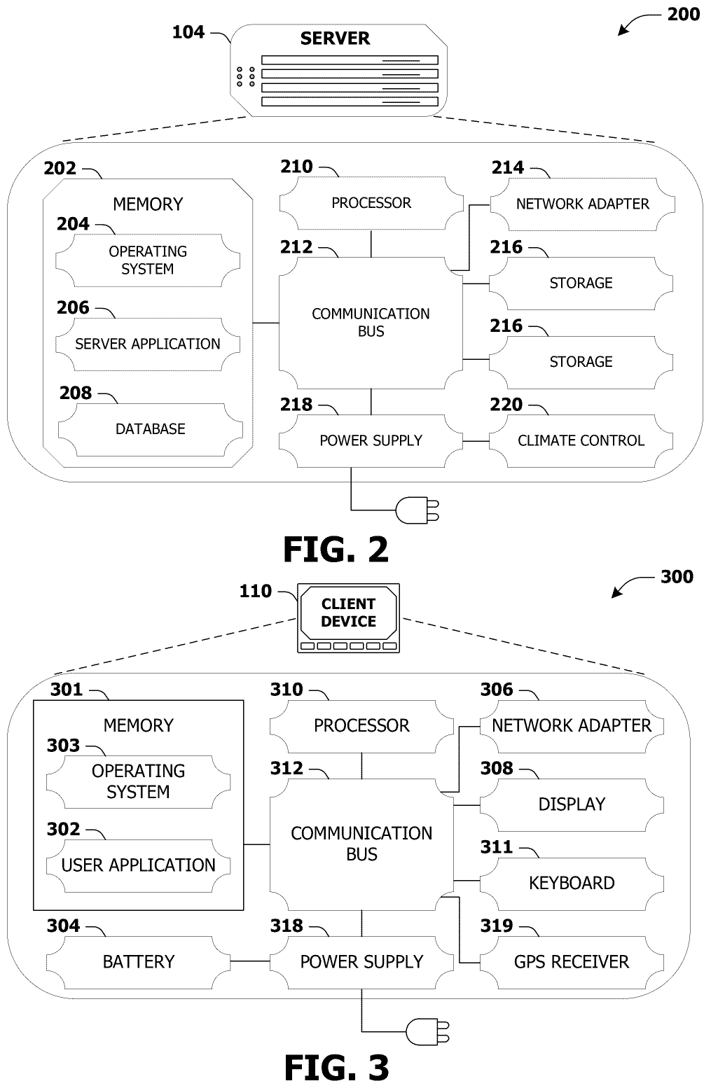

FIG. 2 presents a schematic architecture diagram 200 of a server 104 that may utilize at least a portion of the techniques provided herein. Such a server 104 may vary widely in configuration or capabilities, alone or in conjunction with other servers, in order to provide a service such as the service 102.

The server 104 may comprise a variety of peripheral components, such as a wired and/or wireless network adapter 214 connectible to a local area network and/or wide area network; one or more storage components 216, such as a hard disk drive, a solid-state storage device (SSD), a flash memory device, and/or a magnetic and/or optical disk reader.

The server 104 may comprise memory 202 storing various forms of applications, such as an operating system 204; one or more server applications 206, such as a hypertext transport protocol (HTTP) server, a file transfer protocol (FTP) server, or a simple mail transport protocol (SMTP) server; and/or various forms of data, such as a database 208 or a file system.

The server 104 may comprise one or more processors 210 that process instructions. The one or more processors 210 may optionally include a plurality of cores; one or more coprocessors, such as a mathematics coprocessor or an integrated graphical processing unit (GPU); and/or one or more layers of local cache memory.

The server 104 may comprise a mainboard featuring one or more communication buses 212 that interconnect the processor 210, the memory 202, and various peripherals, using a variety of bus technologies, such as a variant of a serial or parallel AT Attachment (ATA) bus protocol; a Uniform Serial Bus (USB) protocol; and/or Small Computer System Interface (SCI) bus protocol. In a multibus scenario, a communication bus 212 may interconnect the server 104 with at least one other server.

The server 104 may operate in various physical enclosures, such as a desktop or tower, and/or may be integrated with a display as an “all-in-one” device. The server 104 may be mounted horizontally and/or in a cabinet or rack, and/or may simply comprise an interconnected set of components.

The server 104 may provide power to and/or receive power from another server and/or other devices. The server 104 may comprise a dedicated and/or shared power supply 218 that supplies and/or regulates power for the other components. The server 104 may comprise a shared and/or dedicated climate control unit 220 that regulates climate properties, such as temperature, humidity, and/or airflow.

The server 104 may include one or more other components that are not shown in the schematic diagram 200 of FIG. 2, such as a display; a display adapter, such as a graphical processing unit (GPU); input peripherals, such as a keyboard and/or mouse; and a flash memory device that may store a basic input/output system (BIOS) routine that facilitates booting the server 104 to a state of readiness. A plurality of such servers 104 may be configured and/or adapted to utilize at least a portion of the techniques presented herein.

FIG. 3 presents a schematic architecture diagram 300 of a client device 110 whereupon at least a portion of the techniques presented herein may be implemented. Such a client device 110 may vary widely in configuration or capabilities, in order to provide a variety of functionality to a user such as the user 112.

The client device 110 may comprise memory 301 storing various forms of applications, such as an operating system 303; one or more user applications 302, such as document applications, media applications, file and/or data access applications, communication applications such as web browsers and/or email clients, utilities, and/or games; and/or drivers for various peripherals.

In some examples, as a user 112 interacts with a software application on a client device 110 (e.g., an instant messenger and/or electronic mail application), descriptive content in the form of signals or stored physical states within memory (e.g., an email address, instant messenger identifier, phone number, postal address, message content, date, and/or time) may be identified.

In such examples, descriptive content may be stored, typically along with contextual content. For example, the source of an email address (e.g., a communication received from another user via an instant messenger application) may be stored as contextual content associated with the email address. Contextual content, therefore, may identify circumstances surrounding receipt of an email address (e.g., the date or time that the email address was received), and may be associated with descriptive content. Contextual content, may, for example, be used to subsequently search for associated descriptive content. For example, a search for email addresses received from specific individuals, received via an instant messenger application or at a given date or time, may be initiated.

The client device 110 may comprise one or more processors 310 that process instructions. The one or more processors 310 may optionally include a plurality of cores; one or more coprocessors, such as a mathematics coprocessor or an integrated graphical processing unit (GPU); and/or one or more layers of local cache memory.

The client device 110 may comprise a dedicated and/or shared power supply 318 that supplies and/or regulates power for other components, and/or a battery 304 that stores power for use while the client device 110 is not connected to a power source via the power supply 318. The client device 110 may provide power to and/or receive power from other client devices.

The client device 110 may comprise a variety of peripheral components, such as a wired and/or wireless network adapter 306 connectible to a local area network and/or wide area network; one or more output components, such as a display 308 coupled with a display adapter (optionally including a graphical processing unit (GPU)), a sound adapter coupled with a speaker, and/or a printer; input devices for receiving input from the user, such as a keyboard 311, a mouse, a microphone, a camera, and/or a touch-sensitive component of the display 308; and/or environmental sensors, such as a global positioning system (GPS) receiver 319 that detects the location, velocity, and/or acceleration of the client device 110, a compass, accelerometer, and/or gyroscope that detects a physical orientation of the client device 110.

The client device 110 may comprise a mainboard featuring one or more communication buses 312 that interconnect the processor 310, the memory 301, and various peripherals, using a variety of bus technologies, such as a variant of a serial or parallel AT Attachment (ATA) bus protocol; the Uniform Serial Bus (USB) protocol; and/or the Small Computer System Interface (SCI) bus protocol.

The client device 110 may include one or more other components that are not shown in the schematic architecture diagram 300 of FIG. 3, such as one or more storage components, such as a hard disk drive, a solid-state storage device (SSD), a flash memory device, and/or a magnetic and/or optical disk reader; and/or a flash memory device that may store a basic input/output system (BIOS) routine that facilitates booting the client device 110 to a state of readiness. In some examples, the client device 110 may include a climate control unit that regulates climate properties, such as temperature, humidity, and airflow.

The client device 110 may include one or more servers that may locally serve the client device 110 and/or other client devices of the user 112 and/or other individuals. For example, a locally installed webserver may provide web content in response to locally submitted web requests. Many such client devices 110 may be configured and/or adapted to utilize at least a portion of the techniques presented herein.

The client device 110 may serve the user in a variety of roles, such as a workstation, kiosk, media player, gaming device, and/or appliance. The client device 110 may therefore be provided in a variety of form factors, such as a desktop or tower workstation; an “all-in-one” device integrated with a display 308; a laptop, tablet, convertible tablet, or palmtop device; a wearable device mountable in a headset, eyeglass, earpiece, and/or wristwatch, and/or integrated with an article of clothing; and/or a component of a piece of furniture, such as a tabletop, and/or of another device, such as a vehicle or residence.

One or more devices and/or techniques for evaluating images of a brain of a patient and/or determining a functional outcome, a probability of hemorrhage expansion of the patient and/or a treatment plan of the patient are provided. A computed tomography (CT) scan of a brain of a patient may be analyzed to identify one or more anatomic landmarks and/or a hemorrhage region. A normalized hemorrhage map indicative of the hemorrhage region may be generated based upon the one or more anatomic landmarks and the hemorrhage region in the CT scan. A spatial representation of the hemorrhage region may be generated based upon a comparison of the normalized hemorrhage map to a dissimilarity heat map. Using the spatial representation of the hemorrhage region, a functional outcome and/or a treatment plan associated with the patient may be determined with increased accuracy.

In some examples, a brain tissue region that excludes the hemorrhage region may be identified in the CT scan. The brain tissue region and the hemorrhage region may be used to determine a normalized CT-density associated with the hemorrhage region. A probability of hemorrhage expansion may be determined based upon the normalized CT-density. In some examples, the probability of hemorrhage expansion may be determined with reduced cost and/or time spent in comparison with determining the probability by generating a computed tomography angiogram (CTA) of the patient (which may require administering contrast dye into the patient's blood vessels, for example) and analyzing the CTA to determine whether a spot sign is apparent in the CTA. For example, the CT scan comprise a computed tomography of the head (CTH) scan performed without administering the contrast agent, which may enable the probability to determined more quickly and/or in a less expensive manner. Alternatively and/or additionally, a CTA may be performed and/or used in combination with the normalized CT-density to determine the probability with increased accuracy.

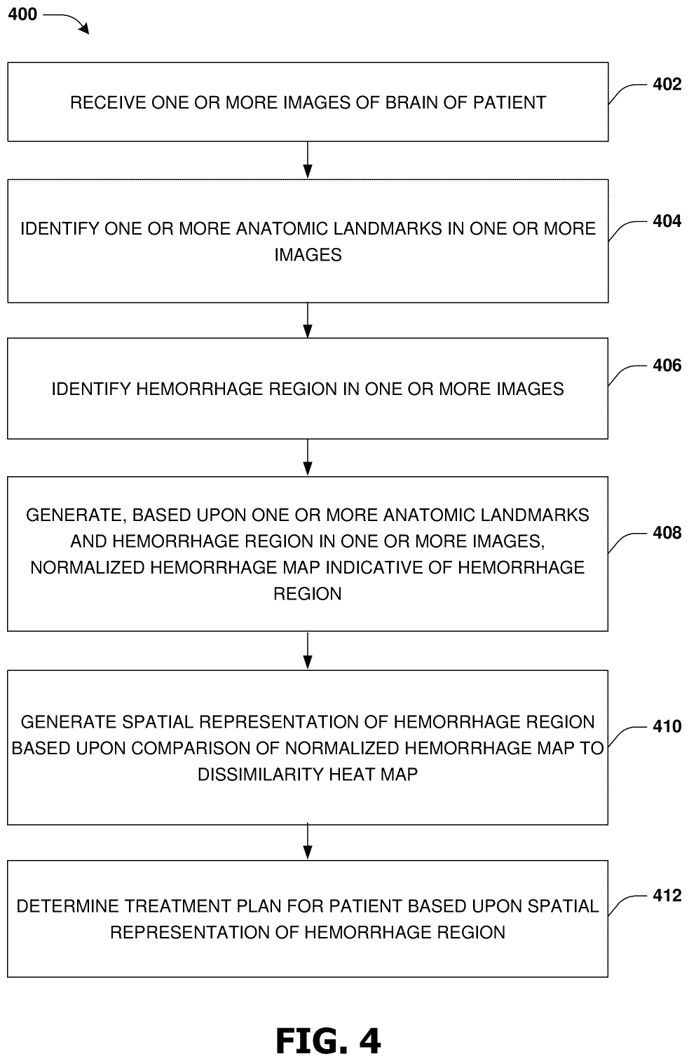

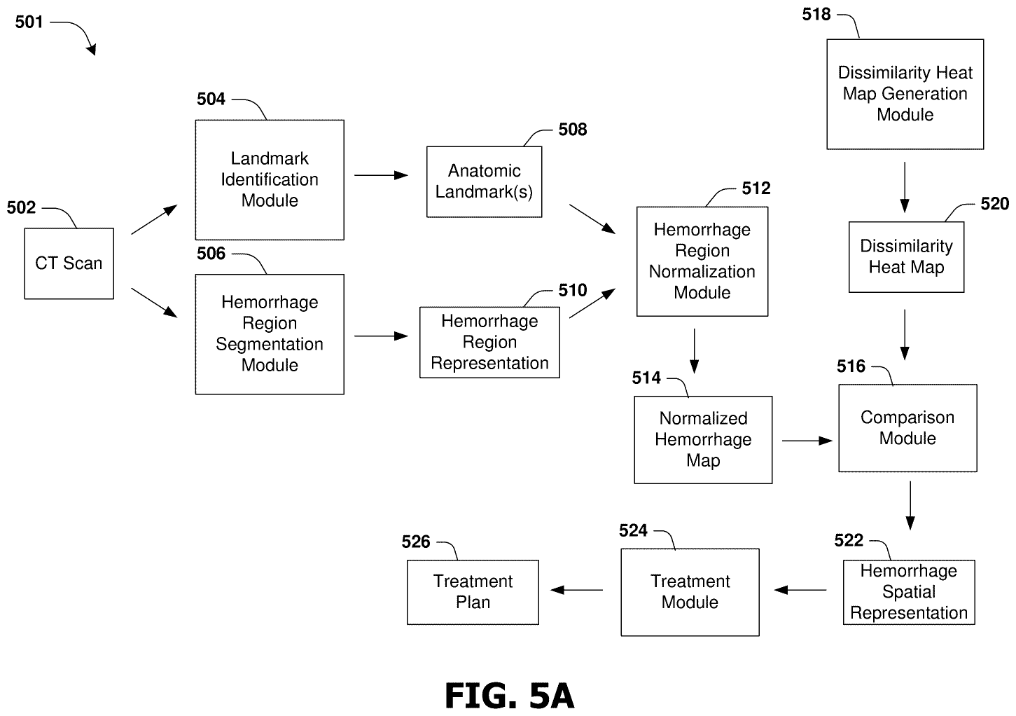

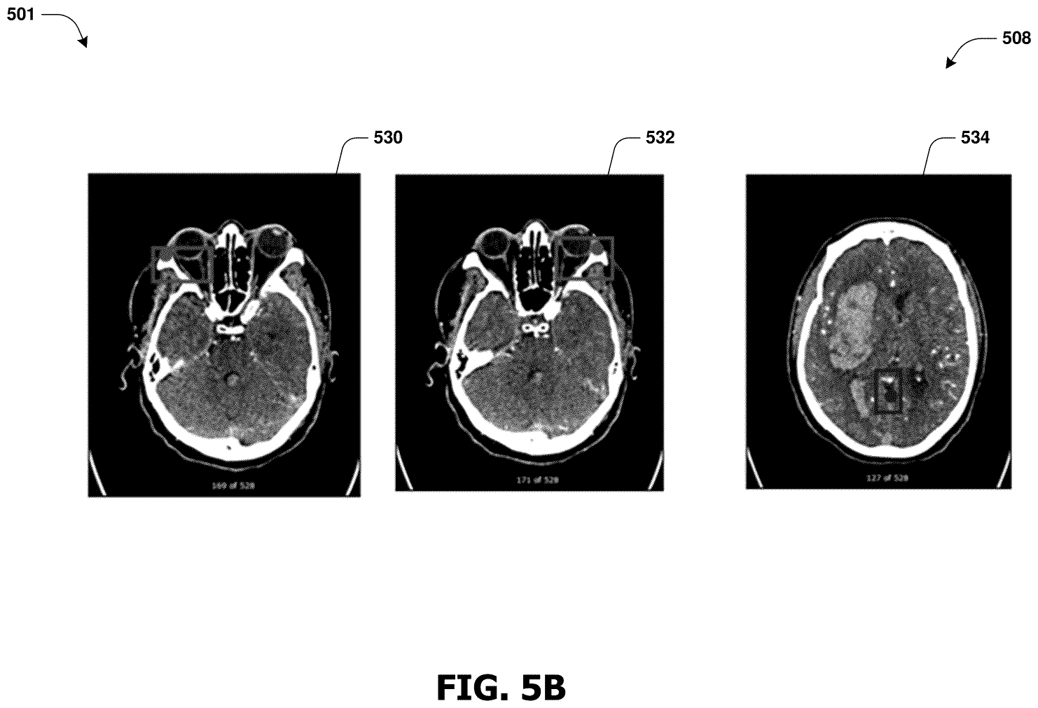

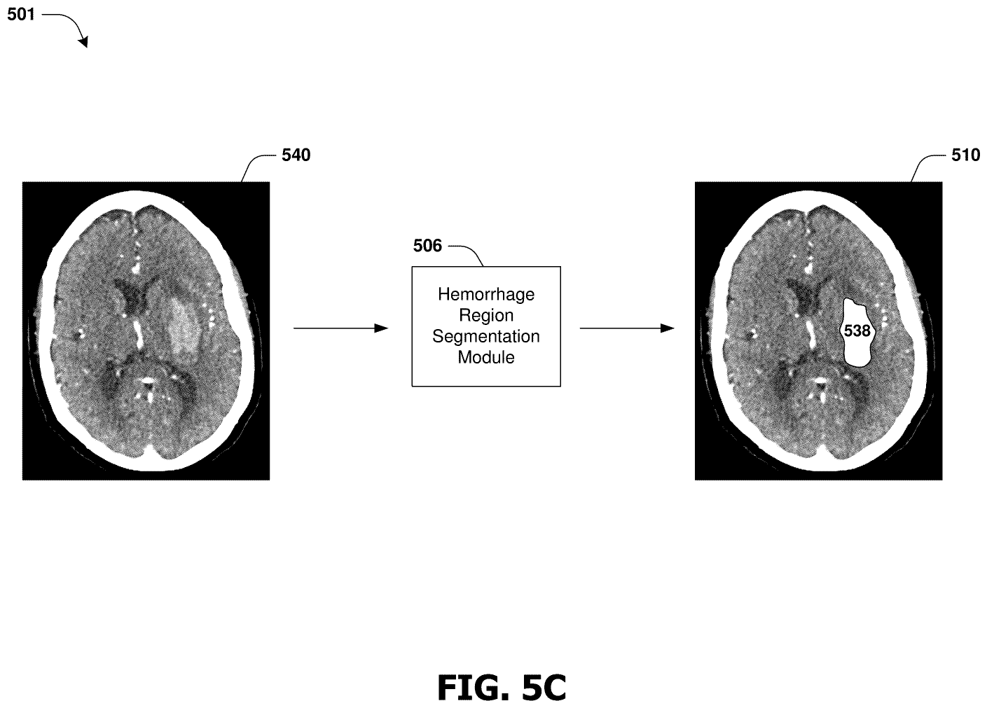

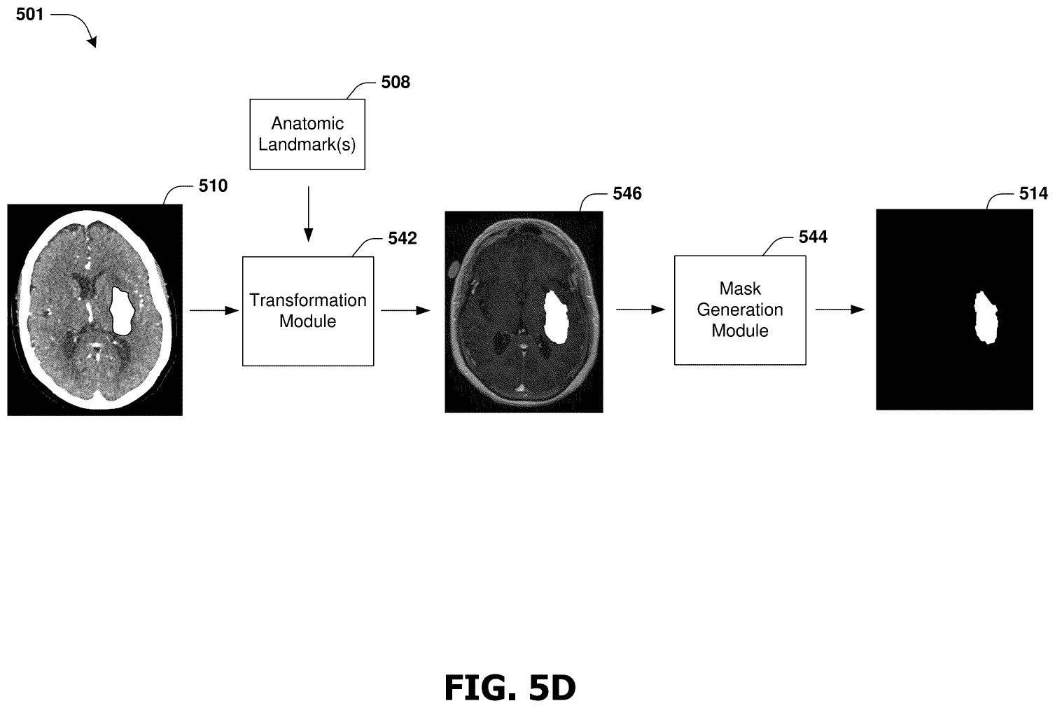

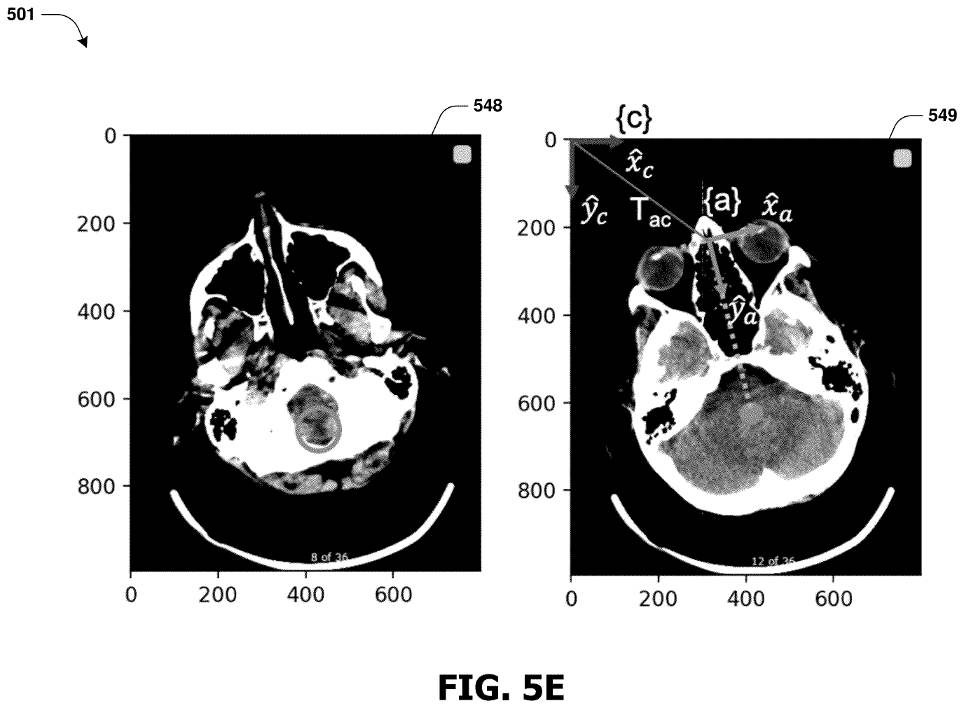

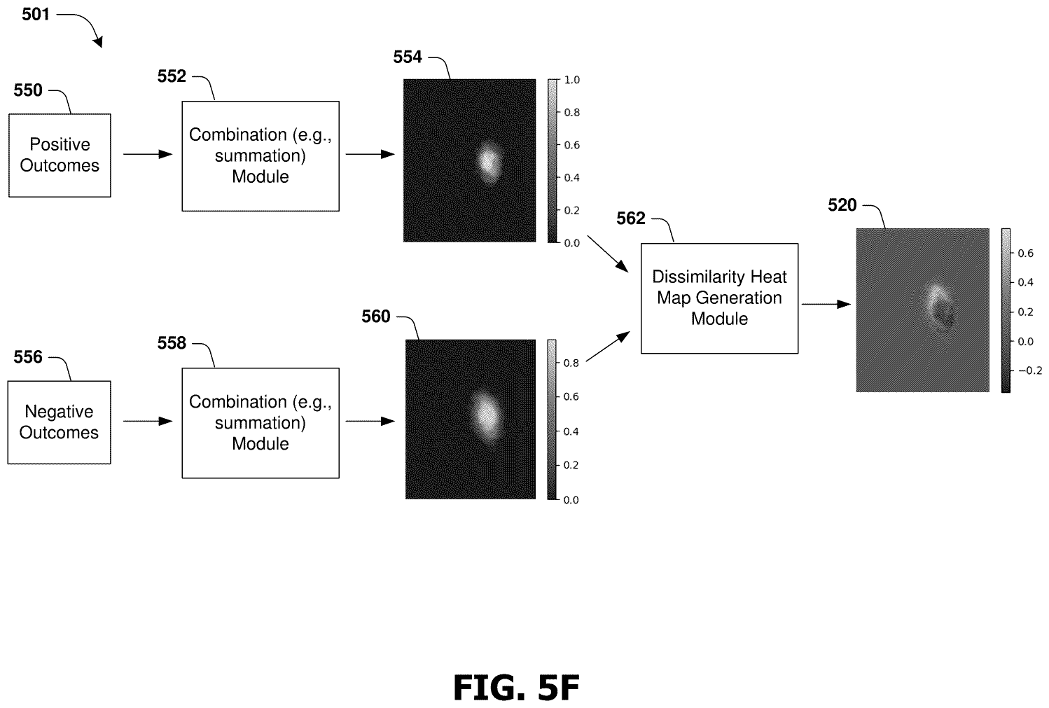

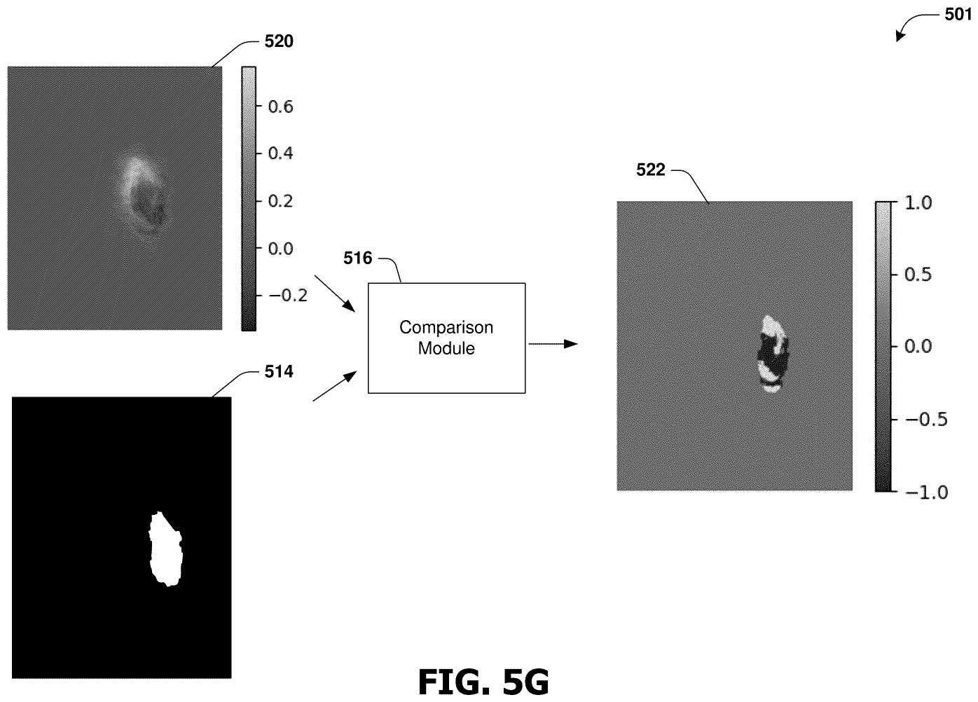

An embodiment of evaluating an image of a brain of a patient and/or determining a functional outcome and/or a treatment plan for the patient (automatically and/or without manual user intervention, for example) is illustrated by an example method 400 of FIG. 4, and is further described in conjunction with system 501 of FIGS. 5A-5G. At 402 of FIG. 4, one or more images of a brain of a patient may be received. It may be appreciated that a patient may be a person (undergoing medical treatment, for example), an animal (undergoing veterinary treatment, for example), etc. In some examples, the one or more images may comprise one or more images of a CT scan 502 (shown in FIG. 5A). For example, the CT scan 502 may comprise cross-sectional images corresponding to various sections of the brain. The sections of the brain represented by images of the CT scan 502 may be referred to as “slices”. In some examples, a slice represented by an image of the CT scan 502 may have a thickness, and/or the image may be representative of data averaged over a volume of tissue having the slice thickness. In some examples, the CT scan 502 may comprise a computed tomography of the head (CTH) scan. In some examples, the CT scan 502 may comprise a computed tomography angiogram (CTA). For example, a contrast agent may be administered (e.g., injected) into blood vessels of the patient, and/or the CT 502 scan may be captured by a CT scanner while the contrast agent passes through the blood vessels. In some examples, the CT scan 502 may comprise a non-contrast CT scan (e.g., a CTH scan without administering the contrast agent). Embodiments are contemplated in which the one or more images are different than CT scan images, such as where the one or more images comprise magnetic resonance imaging (MRI) images, and/or other types of images.

At 404 of FIG. 4, one or more anatomic landmarks in the one or more images may be identified. FIG. 5A illustrates the one or more images (of the CT scan 502, for example) being provided to a landmark identification module 504, which may be used to generate an anatomic landmark dataset 508 indicative of the one or more anatomic landmarks. In some examples, the landmark identification module 504 may use a landmark identification machine learning model to identify the one or more anatomic landmarks and/or generate the anatomic landmark dataset 508. The landmark identification machine learning model may be trained to identify anatomic landmarks in the one or more images using landmark identification training information. For example, the landmark identification training information may comprise a set of images (e.g., CT scan images) associated with a set of patients, and/or label information (e.g., ground truth information) that identifies one or more corresponding anatomic landmarks (e.g., anatomic landmarks to be detected by the landmark identification machine learning model) in the set of images.

FIG. 5B illustrates identification of the one or more anatomic landmarks. In some examples, the one or more anatomic landmarks may comprise a right inferior orbital rim of the patient (and/or other point and/or region of a right orbital rim of the patient), a left inferior orbital rim of the patient (and/or other point and/or region of a left orbital rim of the patient), and/or a posterior tentorial incisura. Other types of landmarks of the one or more anatomic landmarks identified by the anatomic landmark dataset 508 are within the scope of the present disclosure. For example, the one or more anatomic landmarks may comprise a right lens of the patient, a left lens of the patient, a cerebellar tonsils of the patient, and/or one or more other landmarks. The anatomic landmark dataset 508 (output by the landmark identification module 504) may comprise a first anatomic landmark representation 530 identifying the right inferior orbital rim with a red circle, a second anatomic landmark representation 532 identifying the left inferior orbital rim with a green circle, and/or third anatomic landmark representation 532 identifying the posterior tentorial incisura with a blue circle. In some examples, the one or more anatomic landmarks identified by the anatomic landmark dataset 508 may comprise anatomic landmarks identified in different images (representing different volumetric cross-sectional slices, for example) of the CT scan 502.

At 406 of FIG. 4, a hemorrhage region in the one or more images may be identified. For example, the hemorrhage region may comprise an intracranial hemorrhage (ICH) region, a basal ganglia intracranial hemorrhage (bgICH) region, and/or other type of hemorrhage region. FIG. 5A illustrates the one or more images (of the CT scan 502, for example) being provided to a hemorrhage region segmentation module 506, which may be used to generate a hemorrhage region representation 510 indicative of the hemorrhage region. In some examples, the hemorrhage region may be identified based upon the one or more anatomic landmarks indicated by the anatomic landmark dataset 508. For example, the hemorrhage region segmentation module 506 may determine an object in the one or more images is not the hemorrhage region based upon a determination that the object is within a threshold distance (e.g., one or more centimeters) of an anatomic landmark of the one or more anatomic landmarks.

In some examples, the hemorrhage region segmentation module 506 may use a hemorrhage region segmentation machine learning model to identify the hemorrhage region in the one or more images and/or generate the hemorrhage region representation 510 indicative of the hemorrhage region. The hemorrhage region segmentation machine learning model may be trained to identify the hemorrhage region in the one or more images using hemorrhage region segmentation training information. For example, the hemorrhage region segmentation training information may comprise a set of images (e.g., CT scan images) associated with a set of patients, and/or label information (e.g., ground truth information) that identifies hemorrhage regions in the set of images.

FIG. 5C illustrates identification of the hemorrhage region in the one or more images. In some examples, an image 540 of the CT scan 502 may be provided to the hemorrhage region segmentation module 506. The hemorrhage region segmentation module 506 may analyze the image 540 (using the hemorrhage region segmentation machine learning model, for example) to identify the hemorrhage region in the image 540 and/or may generate the hemorrhage region representation 510 to indicate of a segment 538, of the image 540, that corresponds to the hemorrhage region. For example, the segment 538 may be set to white (as shown in FIG. 5C), black, or other color to indicate a position and/or boundaries of the hemorrhage region. In some examples, the segment 538 identified by the hemorrhage region segmentation module 506 may include a parenchymal component of the hemorrhage region and/or may exclude an intraventricular component of the hemorrhage region.

At 408 of FIG. 4, a normalized hemorrhage map 514 (shown in FIGS. 5A, 5D, and 5G) indicative of the hemorrhage region may be generated based upon the one or more anatomic landmarks (indicated by the anatomic landmark dataset 508, for example) and/or the hemorrhage region (indicated by the hemorrhage region representation 510, for example) in the one or more images. FIG. 5A illustrates the anatomic landmark dataset 508 and/or the hemorrhage region representation 510 being provided to a hemorrhage region normalization module 512, which may be used to generate a hemorrhage region representation 510 indicative of the hemorrhage region. For example, coordinates of the hemorrhage region may be mapped to coordinates of a stereotactic reference frame (e.g., a stereotactic and/or anatomic coordinate system) based upon the one or more anatomic landmarks.

FIG. 5D illustrates generation of the normalized hemorrhage map 514. In some examples, the hemorrhage region normalization module 512 (shown in FIG. 5C) may comprise a transformation module 542 for transforming the hemorrhage region representation 510 to a normalized hemorrhage region representation 546 and/or a mask generation module 544 for generating the normalized hemorrhage map 514 (which may comprise a mask indicating a normalized hemorrhage region, for example) based upon the normalized hemorrhage region representation 546. In some examples, the transformation module 542 may define the stereotactic reference frame based upon the one or more anatomic landmarks, determine one or more relationships between the stereotactic reference frame and an image coordinate frame (e.g., a coordinate system of the CT scan 502 and/or a CT scanner used to generate the CT scan 502) of the hemorrhage region representation 510, generate a transformation profile (e.g., a transformation matrix) based upon the one or more relationships, and/or apply the transformation profile (to the hemorrhage region representation 510, for example) to generate the normalized hemorrhage region representation 546.

FIG. 5E illustrates aspects of the stereotactic reference frame (labeled {a} in FIG. 5E) and the image coordinate frame (labeled {c} in FIG. 5E). In some examples, the image coordinate frame {c} and the stereotactic reference frame {a} are depicted in a representation 549 of an image of the CT scan 502. In some examples, the image coordinate frame {c} has axes \hat{x}_{c}, ŷ_{c} (shown with red arrows in FIG. 5E), and \hat{z}_{c} (not shown in FIG. 5E). The stereotactic reference frame {a} has axes \hat{x}ₐ, ŷₐ (shown with green arrows in FIG. 5E), and \hat{z}ₐ (not shown in FIG. 5E). In some examples, the image coordinate frame {c} may be used to define the axes \hat{x}ₐ, ŷₐ, and \hat{z}ₐ of the stereotactic reference frame {a}. Dashed green lines in the representation 549 demonstrate projected intersections of the axes of the stereotactic reference frame {a} with anatomic landmarks, such as an intersection of axis \hat{x}ₐ with a left anterior anatomic landmark (e.g., the left inferior orbital rim and/or the left lens) of the one or more anatomic landmarks (indicated by the anatomic landmark dataset 508, for example), and/or an intersection of axis ŷₐ with a posterior anatomic landmark (e.g., the posterior tentorial incisura and/or the cerebellar tonsils) of the one or more anatomic landmarks.

In some examples, the axis \hat{x}ₐ may be defined based upon a first set of landmarks (e.g., two anterior landmarks) of the one or more anatomic landmarks (indicated by the anatomic landmark dataset 508, for example). For example, the first set of landmarks used to define the axis \hat{x}ₐ may comprise the right inferior orbital rim and the left inferior orbital rim, such as where the axis \hat{x}ₐ is defined to overlap with and/or extend along a line between the right inferior orbital rim and the left inferior orbital rim. In some examples, the first set of landmarks used to define the axis \hat{x}ₐ may comprise the right lens and the left lens, such as where the axis \hat{x}ₐ is defined to overlap with and/or extend along a line between the right lens and the left lens.

In some examples, the axis ŷₐ may be defined based upon the posterior anatomic landmark (a location of which is depicted with a closed green circle in the representation 549) of the one or more anatomic landmarks and/or a landmark point determined based upon the first set of landmarks. In the representation 549 and representation 548, the posterior anatomic landmark may comprise the cerebellar tonsils (and/or a most inferior point of the cerebellar tonsils). Embodiments are contemplated in which the posterior anatomic landmark comprises the posterior tentorial incisura or other landmark. For example, the landmark point may correspond to a point (e.g., midpoint) along a line between the first set of landmarks (e.g., the line between the right inferior orbital rim and the left inferior orbital rim or the line between the right lens and the left lens). In some examples, the axis ŷₐ may be defined to overlap with and/or extend along a line between the landmark point and the posterior anatomic landmark.

In some examples, the posterior anatomic landmark (e.g., the cerebellar tonsils) is located inferior to the first set of landmarks. For example, the posterior anatomic landmark (e.g., the cerebellar tonsils) is depicted in a green circle shown in the representation 548, which may comprise an image, of the CT scan 502, that is inferior to the image of the CT scan 502 depicted in the representation 549 (e.g., an axial slice represented by the representation 548 where the cerebellar tonsils is depicted is inferior to an axial slice represented by the representation 549 where the first set of landmarks and the location of the cerebellar tonsils are depicted). In some examples, based upon the posterior anatomic landmark being located inferior to the first set of landmarks, the axis ŷₐ may be defined to extend in an angled inferior direction that extends from the landmark point in the axial slice represented by the representation 549 to the (inferior) axial slice represented by the representation 548.

In some examples, the axis \hat{z}ₐ is defined based upon the first set of landmarks and/or a rostral direction. For example, the axis \hat{z}ₐ may be defined as extending orthogonal to the line between the first set of landmarks (e.g., the line between the right inferior orbital rim and the left inferior orbital rim or the line between the right lens and the left lens) and/or in the rostral direction (e.g., the axis \hat{z}ₐ may have rostral-caudal directionality).

In some examples, in response to determining (by the transformation module 542, for example) the axes of the stereotactic reference frame {a}, the transformation module 542 may determine the one or more relationships based upon a comparison of the stereotactic reference frame {a} to the image coordinate frame {c}. In some examples, the one or more relationships may comprise a translation from the image coordinate frame {c} to the stereotactic reference frame {a}, which may correspond to a translation from an origin of the image coordinate frame {c} (e.g., a top left corner of the representation 549) to an origin of the stereotactic reference frame {a} (e.g., the landmark point determined based upon the first set of landmarks). In some examples, the one or more relationships may comprise one or more angles of rotation from axes of the image coordinate frame {c} to axes of the stereotactic reference frame {a}, such as an angle of rotation from the axis ŷ_{c} of the image coordinate frame {c} to the axis ŷₐ of the stereotactic reference frame {a}, an angle of rotation from the axis \hat{x}_{c} of the image coordinate frame {c} to the axis \hat{x}ₐ of the stereotactic reference frame {a}, and/or an angle of rotation from the axis \hat{z}_{c} of the image coordinate frame {c} to the axis \hat{z}ₐ of the stereotactic reference frame {a}.

In some examples, the transformation module 542 may generate the transformation profile (e.g., a transformation matrix) based upon the one or more relationships, such as based upon the translation from the image coordinate frame {c} to the stereotactic reference frame {a} and/or the one or more angles of rotation from axes of the image coordinate frame {c} to axes of the stereotactic reference frame {a}. A blue line in the representation 549 depicts the transformation profile converting a coordinate of the image coordinate frame {c} (e.g., the origin of the image coordinate frame {c}) to a coordinate of the stereotactic reference frame {a} (e.g., the origin of the stereotactic reference frame {a}).

In some examples, the transformation module 542 may apply the transformation profile to the hemorrhage region representation 510 to generate the normalized hemorrhage region representation 546. In some examples, pixels of the normalized hemorrhage region representation 546 that correspond to the hemorrhage region may be set to white (as shown in FIG. 5D), black, or other color to indicate a position and/or boundaries of a normalized version of the hemorrhage region (that can be compared with other hemorrhage regions associated with other patients, for example). In some examples, the mask generation module 544 may generate the normalized hemorrhage map 514 to comprise a binary mask with a first color (e.g., white) for coordinates within the hemorrhage region (e.g., coordinates within the normalized version of the hemorrhage region) and/or a second color (e.g., black) for coordinates outside the hemorrhage region (e.g., coordinates outside the normalized version of the hemorrhage region).

In some examples, the one or more anatomic landmarks may comprise the right inferior orbital rim (ro), the left inferior orbital rim (lo) and the posterior tentorial incisura (ti). Pixel coordinates were converted to metric units (and/or other units) using a scalar conversion coordinate, dots per millimeter, in the x- and y-axes and/or using CT-axial slice height in the z-axis. The angle formed between vectors a and x may be defined as follows:

$$θ = {cos}^{- 1} \frac{a \cdot x}{\vert a \vert \vert x \vert} ,$$

where a and x may be the vectors formed from ro to ti and ro to lo, respectively. The origin may be defined as the point o on vector x, at which a vector drawn between o and ti may be orthogonal to x. The distance d between the origin o and ro may be defined as:
d=a*cos θ. The origin o at a distance d along vector x may be defined using the following vector operations:

$$o = ro + \frac{d}{\vert x \vert} x .$$

Unit vectors \hat{x}, ŷ, and \hat{z} may be defined in the anatomic coordinate space such that \hat{x} defined medial-lateral directionality, ŷ defined anterior-posterior directionality, and/or \hat{z} defined rostral-caudal directionality. All were defined starting from the origin o. Unit vector \hat{x} may have the same directionality of vector x. Unit vector ŷ may be defined by o in the direction of the vector spanning from o to ti. Unit vector \hat{z} may be defined as the cross product between \hat{x} and ŷ. The transformation matrix (e.g., the transformation profile) for converting coordinates between the pixel coordinate space (e.g., the image coordinate frame {c}) and the anatomic coordinate space (e.g., the stereotactic reference frame {a}), Tₐₚ, may be defined as:

$${T}_{ap} = [ \begin{matrix}{R}^{T} & - {R}^{T} o \\ 0 & 1\end{matrix} ] ,$$

where R is the rotation matrix formed by \hat{x}, ŷ, and \hat{z}, and R^{T} represents the rotation matrix transposed. Transformation of coordinates c in the pixel coordinate space to the anatomic space may be performed as: cₐ=Tₐₚ·cₚ. Transformation of coordinates back to the pixel coordinate system for visual representation may be performed using Tₚₐ, which may be the inverse of Tₐₚ as follows:

$${T}_{pa} = {T}_{ap}^{- 1} = [ \begin{matrix}R & o \\ 0 & 1\end{matrix} ] .$$

Individual bgICH masks may be transformed from their own pixel coordinate spaces into a single pixel coordinate space (to generate a dissimilarity heat map 520, for example). The transformation from each of the individual patient pixel coordinate spaces p₁ to another universal pixel coordinate space p₂ may be performed through the common anatomic coordinate space using the properties of matrix operations as follows: Tₚ₂ₚ₁=Tₚ₂ₐ·Tₐₚ₁ such that coordinates in p₁ may be converted to coordinates in p₂ as follows:

$${c}_{2} = {T}_{p 2 p 1} {c}_{1} .$$

At 410 of FIG. 4, a spatial representation 522 (shown in FIG. 5A and FIG. 5G) of the hemorrhage region may be generated based upon a comparison of the normalized hemorrhage map 514 to a dissimilarity heat map 520 (shown in FIGS. 5A, 5F, and 5G). FIG. 5A illustrates the dissimilarity heat map 520 and the normalized hemorrhage map 514 to a comparison module 516, which may be used to generate the spatial representation 522 of the hemorrhage region. In some examples, the dissimilarity heat map 520 may be generated using a dissimilarity heat map generation module 518.

FIG. 5F illustrates generation of the dissimilarity heat map 520 using the dissimilarity heat map generation module 518. In some examples, the dissimilarity heat map 520 may be generated based upon a comparison of a first set of hemorrhage maps 550 associated with a first group of patients to a second set of hemorrhage maps 556 associated with a second group of patients. In some examples, the first set of hemorrhage maps 550 and/or the first group of patients may be associated with positive (and/or favorable and/or functional) outcomes and/or the second set of hemorrhage maps 556 and/or the second group of patients may be associated with negative (and/or poor and/or severely disabled) outcomes.

In some examples, modified Rankin Scale (mRS)-based categorization may be performed to determine the first set of hemorrhage maps 550 and/or the second set of hemorrhage maps 556. For example, the mRS-based categorization may comprise determining mRS scores associated with a plurality of hemorrhage maps and/or patients associated with the plurality of hemorrhage maps, and/or grouping hemorrhage maps of the plurality of hemorrhage maps into the first set of hemorrhage maps 550 and/or the second set of hemorrhage maps 556 based upon the mRS scores. For example, a first hemorrhage map of the plurality of hemorrhage maps may be included in the first set of hemorrhage maps based upon a first mRS score associated with the first hemorrhage map, and/or a second hemorrhage map of the plurality of hemorrhage maps may be included in the second set of hemorrhage maps based upon a second mRS score associated with the second hemorrhage map. For example, the first hemorrhage map may be included in the first set of hemorrhage maps based upon the first mRS score meeting a threshold mRS score (e.g., being at most 3, such as being 1, 2, or 3). The second hemorrhage map may be included in the first set of hemorrhage maps based upon the first mRS score not meeting the threshold mRS score (e.g., being greater than 3, such as being 4, 5, or 6).

In some examples, an mRS score meeting the threshold mRS score (e.g., mRS being at most 3) may indicate that a patient associated with the mRS score is associated with a favorable and/or functional outcome, that the patient may be independent and/or moderately disabled, that the patient may be mobile without full-time care, that the patient may require help with some daily tasks, and/or that the patient can function in society. In some examples, an mRS score not meeting the threshold mRS score (e.g., mRS being greater than 3) may indicate that a patient associated with the mRS score is associated with a poor outcome, that the patient may be severely disabled, that the patient may be bedridden, and/or that the patient cannot live independently.

In some examples, some or all of hemorrhage maps of the first set of hemorrhage maps 550 and/or the second set of hemorrhage maps 556 may be normalized (to corresponding stereotactic reference frames, for example) using one or more of the techniques provided herein with respect to normalizing the hemorrhage region representation 510 to generate the normalized hemorrhage region representation 546 and/or the normalized hemorrhage map 514. In some examples, some or all of hemorrhage maps of the first set of hemorrhage maps 550 and/or the second set of hemorrhage maps 556 may comprise binary masks with the first color (e.g., white) for coordinates within corresponding (normalized) hemorrhage regions and/or the second color (e.g., black) for coordinates outside corresponding (normalized) hemorrhage regions.

In some examples, the first set of hemorrhage maps 550 may be combined using a first combination module 552 to generate a first heat map 554 associated with positive (and/or favorable and/or functional) outcomes. In some examples, the first combination module 552 may sum the first set of hemorrhage maps 550 to generate the first heat map 554. In some examples, the first heat map 554 may visualize a proportion of hemorrhage region presence, such as where yellow is representative of about 100% hemorrhage region presence at a coordinate and/or where purple is representative of about 0% hemorrhage region presence at a coordinate.

In some examples, the second set of hemorrhage maps 556 may be combined using a second combination module 558 (and/or the first combination module 552) to generate a second heat map 560 associated with negative (and/or poor and/or severely disabled) outcomes. In some examples, the second combination module 558 (and/or the first combination module 552) may sum the second set of hemorrhage maps 556 to generate the second heat map 560. In some examples, the second heat map 560 may visualize a proportion of hemorrhage region presence, such as where yellow is representative of about 100% hemorrhage region presence at a coordinate and/or where purple is representative of about 0% hemorrhage region presence at a coordinate.

In some examples, the first heat map 554 and/or the second heat map 560 may be provided to a dissimilarity heat map generation module 562, which may generate the dissimilarity heat map 520 based upon a comparison of the first heat map 554 to the second heat map 560. For example, the dissimilarity heat map generation module 562 may subtract the second heat map 560 (associated with negative outcomes) from the first heat map 554 (associated with positive outcomes) to generate the dissimilarity heat map 520. In some examples, the dissimilarity heat map 520 is representative of disproportionate location representation according to outcome state (e.g., positive outcome versus negative outcome). In some examples, coordinates represented by negative numbers in the dissimilarity heat map 520 may be indicative of a greater proportion of positive outcomes (and/or a lesser proportion of negative outcomes). In some examples, coordinates represented by positive numbers in the dissimilarity heat map 520 may be indicative of a lesser proportion of positive outcomes (and/or a greater proportion of negative outcomes).

FIG. 5G illustrates generation of the spatial representation 522 of the hemorrhage region using the comparison module 516 based upon the dissimilarity heat map 520 and/or the normalized hemorrhage map 514. In some examples, the comparison module 516 may combine the normalized hemorrhage map 514 with the dissimilarity heat map 520 to generate the spatial representation 522 of the hemorrhage region. For example, the comparison module 516 may multiply the normalized hemorrhage map 514 with the dissimilarity heat map 520 to generate the spatial representation 522 of the hemorrhage region. In some examples, yellow (and/or a different color) may be representative of overlap of the normalized hemorrhage map 514 with negative functional outcome disproportionate representation, and/or blue (and/or a different color) may be representative of overlap of the normalized hemorrhage map 514 with positive functional outcome disproportionate representation. In some examples, the spatial representation 522 of the hemorrhage region may be representative of (and/or may be usable to determine) a first differential volume overlap associated with the patient. In some examples, the first differential volume overlap associated with the patient may be determined by subtracting volume overlap with negative values from volume overlap with positive values in the dissimilarity heat map 520.

In some examples, one or more of the techniques provided herein for generating the normalized hemorrhage map 514 and/or using the normalized hemorrhage map 514 to generate the spatial representation 522 of the hemorrhage region may be performed (e.g., repeated) for each of a plurality of axial slices of the CT scan 502 to generate a plurality of spatial representations of the hemorrhage region (comprising the spatial representation 522) associated with the plurality of axial slices of the CT scan 502. For example, the plurality of axial slices may comprise some or all axial slices of the CT scan 502. In some examples, the spatial representation 522 of the hemorrhage region may be representative of (and/or may be usable to determine) a second differential volume overlap (e.g., total differential volume overlap) associated with the patient.

At 412 of FIG. 4, a treatment plan 526 for the patient may be determined based upon the spatial representation 522 of the hemorrhage region. FIG. 5A illustrates the spatial representation 522 of the hemorrhage region (and/or the plurality of spatial representations) being provided to a treatment module 524, which may be used to generate the treatment plan 526. In some examples, the treatment module 524 may determine the first differential volume overlap based upon the spatial representation 522 of the hemorrhage region, and/or may determine the treatment plan 526 based upon the first differential volume overlap. In some examples, the treatment module 524 may determine the second differential volume overlap based upon the plurality of spatial representations of the hemorrhage region, and/or may determine the treatment plan 526 based upon the second differential volume overlap.

In some examples, the treatment module 524 may determine a first functional outcome associated with the patient based upon the spatial representation 522, the plurality of spatial representations, the first differential volume overlap and/or the second differential volume overlap. Alternatively and/or additionally, other information associated with the patient may be used by the treatment module 524 to determine the first functional outcome, such as at least one of an age of the patient, a stroke score (e.g., a National Institutes of Health Stroke Scale (NIHSS) score), an intraventricular hemorrhage (IVH) extent score (e.g., a modified Graeb scale (mGS) score), and/or other information associated with the patient. In some examples, the treatment module 524 may determine the treatment plan 526 based upon the first functional outcome.

In some examples, the first functional outcome may be indicative of a first probability that the patient would respond positively to (and/or benefit from) a first type of treatment (e.g., surgical hemorrhage evacuation), would have a positive functional outcome (e.g., a functional outcome associated with an mRS score of 1, 2, or 3) after and/or due to undergoing the first type of treatment, and/or would not have a negative functional outcome (e.g., a functional outcome associated with an mRS score of 4, 5, or 6) after and/or due to undergoing the first type of treatment. In some examples, the first functional outcome may be indicative of a second probability that the patient would respond negatively to the first type of treatment (e.g., surgical hemorrhage evacuation), would have a negative functional outcome (e.g., a functional outcome associated with an mRS score of 4, 5, or 6) after and/or due to undergoing the first type of treatment, and/or would not have a positive functional outcome (e.g., a functional outcome associated with an mRS score of 1, 2, or 3) after and/or due to undergoing the first type of treatment. In some examples, the first functional outcome may be indicative of a prediction of whether the first type of treatment is suitable and/or beneficial for the patient. In some examples, increased differential volume overlap may be associated with an increased probability of a negative functional outcome. For example, the first probability may be increased (and/or the second probability may be decreased) based upon an increase of a differential volume overlap (indicated by the spatial representation 522, the plurality of spatial representations, the first differential volume overlap and/or the second differential volume overlap) associated with the patient being greater.

In some examples, the first functional outcome may be indicative of a third probability that the patient would benefit from a second type of treatment (e.g., non-surgical medical treatment), would have a positive functional outcome (e.g., a functional outcome associated with an mRS score of 1, 2, or 3) after and/or due to undergoing the second type of treatment, and/or would not have a negative functional outcome (e.g., a functional outcome associated with an mRS score of 4, 5, or 6) after and/or due to undergoing the second type of treatment. In some examples, the first functional outcome may be indicative of a fourth probability that the patient would respond negatively to the second type of treatment (e.g., non-surgical medical treatment), would have a negative functional outcome (e.g., a functional outcome associated with an mRS score of 4, 5, or 6) after and/or due to undergoing the second type of treatment, and/or would not have a positive functional outcome (e.g., a functional outcome associated with an mRS score of 1, 2, or 3) after and/or due to undergoing the second type of treatment. In some examples, the first functional outcome may be indicative of a prediction of whether the second type of treatment is suitable and/or beneficial for the patient.

In some examples, the first functional outcome may be determined using a functional outcome machine learning model. In some examples, the functional outcome machine learning model may comprise a first multivariate logistic regression model. The functional outcome machine learning model may be trained to determine functional outcomes for patients using functional outcome determination training information. For example, the functional outcome determination training information may comprise sets of patient information associated with a plurality of patients, and/or label information (e.g., ground truth information) that is indicative of sets of functional outcome information associated with the plurality of patients.

A set of patient information (indicated by the functional outcome determination training information) associated with a first patient may be indicative of one or more first spatial representations of a first hemorrhage region of the first patient (which may be generated using one or more of the techniques provided herein with respect to generating the spatial representation 522 and/or the plurality of spatial representations, for example), a differential overlap associated with the first patient (which may be determined using one or more of the techniques provided herein with respect to determining the first differential volume overlap and/or the second differential volume overlap), an age of the first patient, a stroke score (e.g., a NIHSS score) associated with the first patient, an IVH extent score (e.g., a modified Graeb scale (mGS) score) associated with the first patient, and/or other information associated with the first patient.

A set of functional outcome information (indicated by the label information of the functional outcome determination training information) associated with the first patient may be indicative of a type of treatment performed on the first patient (e.g., the first type of treatment and/or the second type of treatment), a functional outcome associated with the first patient after and/or due to undergoing the type of treatment, a change in functional outcome of the first patient from before undergoing the type of treatment to after undergoing the type of treatment (which may indicate whether the type of treatment improved, worsened or had minimal impact on the first patient's health and/or functional outcome), and/or other information associated with the first patient.

In some examples, the treatment plan 526 may be generated to indicate treating the patient with the first type of treatment (e.g., surgical hemorrhage evacuation) based upon the first probability (associated with positive outcome of the first type of treatment, for example) being greater than a first probability threshold and/or based upon the second probability (associated with negative outcome of the first type of treatment, for example) being less than a second probability threshold. Alternatively and/or additionally, the treatment plan 526 may be generated to be indicative of not treating the patient with the first type of treatment (e.g., surgical hemorrhage evacuation) based upon the first probability being less than the first probability threshold and/or based upon the second probability being greater than the second probability threshold.

In some examples, the treatment plan 526 may be generated to indicate treating the patient with the second type of treatment (e.g., non-surgical medical treatment) based upon the first probability (associated with positive outcome of the first type of treatment, for example) being less than the first probability threshold and/or based upon the second probability (associated with negative outcome of the first type of treatment, for example) being greater than the second probability threshold. Alternatively and/or additionally, the treatment plan 526 may be generated to indicate treating the patient with the second type of treatment (e.g., non-surgical medical treatment) based upon the third probability (associated with positive outcome of the second type of treatment) being greater than a third probability threshold and/or based upon the fourth probability (associated with negative outcome of the second type of treatment, for example) being less than a fourth probability threshold. Alternatively and/or additionally, the treatment plan 526 may be generated to be indicative of not treating the patient with the second type of treatment (e.g., non-surgical medical treatment) based upon the third probability being less than the third probability threshold and/or based upon the fourth probability being greater than the fourth probability threshold.

In some examples, the treatment plan 526 may be provided to a client device for display. The client device (and/or the treatment plan 526 displayed on the client device) may be accessible to at least one of the patient, a healthcare professional (e.g., physician, surgeon, nurse, etc.) associated with the patient, etc. In some examples, one or more treatments (e.g., the first type of treatment and/or the second type of treatment) indicated by the treatment plan 526 may be performed automatically (and/or without user intervention) and/or with supervision and/or guidance of the healthcare professional. Alternatively and/or additionally, the treatment plan 526 may comprise a guide for performing the one or more treatments, which may be used by the healthcare professional (and/or a healthcare machine and/or robot) to perform the one or more treatments.

In some examples, the one or more treatments may comprise a surgical hemorrhage evacuation (e.g., the first type of treatment). For example, the surgical hemorrhage evacuation may be scheduled for the patient and/or performed on the patient in response to the treatment plan 526 indicating the surgical hemorrhage evacuation. In some examples, the surgical hemorrhage evacuation (which may also be referred to as surgical hematoma evacuation) may comprise minimally invasive surgery (MIS) for hemorrhage evacuation.

In some examples, the one or more treatments may comprise a non-surgical medical treatment (e.g., the second type of treatment). For example, the non-surgical medical treatment may be administered to the patient in response to the treatment plan 526 indicating the non-surgical medical treatment. In some examples, the non-surgical medical treatment may comprise a blood pressure control treatment to control a blood pressure of the patient, which may comprise administering one or more blood pressure control medicines (e.g., calcium channel blockers, beta-adrenergic receptor antagonists, and/or dihydropyridine derivatives) to the patient, and/or may reduce the risk of further bleeding without reducing cerebral perfusion. In some examples, the non-surgical medical treatment may comprise an anticoagulation reversal treatment to reverse anticoagulation (if present, for example), which may comprise administering one or more anticoagulation reversal medicines (e.g., at least one of phytonadione, protamine sulfate, idarucizumab, andexanet alfa, prothrombin complex concentrate, etc.), and/or may improve and/or restore clotting to mitigate and/or stop active bleeding. In some examples, the non-surgical medical treatment may comprise an intracranial pressure management treatment, which may reduce intracranial pressure. In some examples, the non-surgical medical treatment may comprise providing one or more supportive care treatments such as at least one of ventilatory support (e.g., by endotracheal intubation and mechanical ventilation), fluid balance maintenance (e.g., with intravenous sodium chloride infusion), using one or more antiepileptic medicines to mitigate and/or prevent seizures, using one or more mechanical compression devices and/or anticoagulant prophylaxis to mitigate and/or prevent deep vein thrombosis, etc.

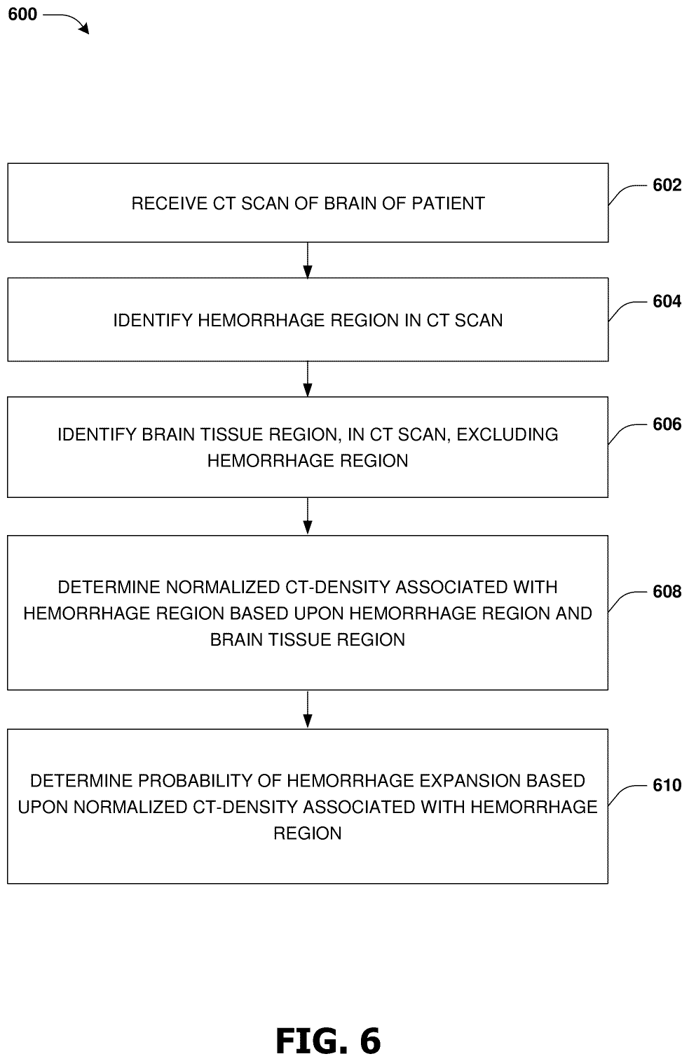

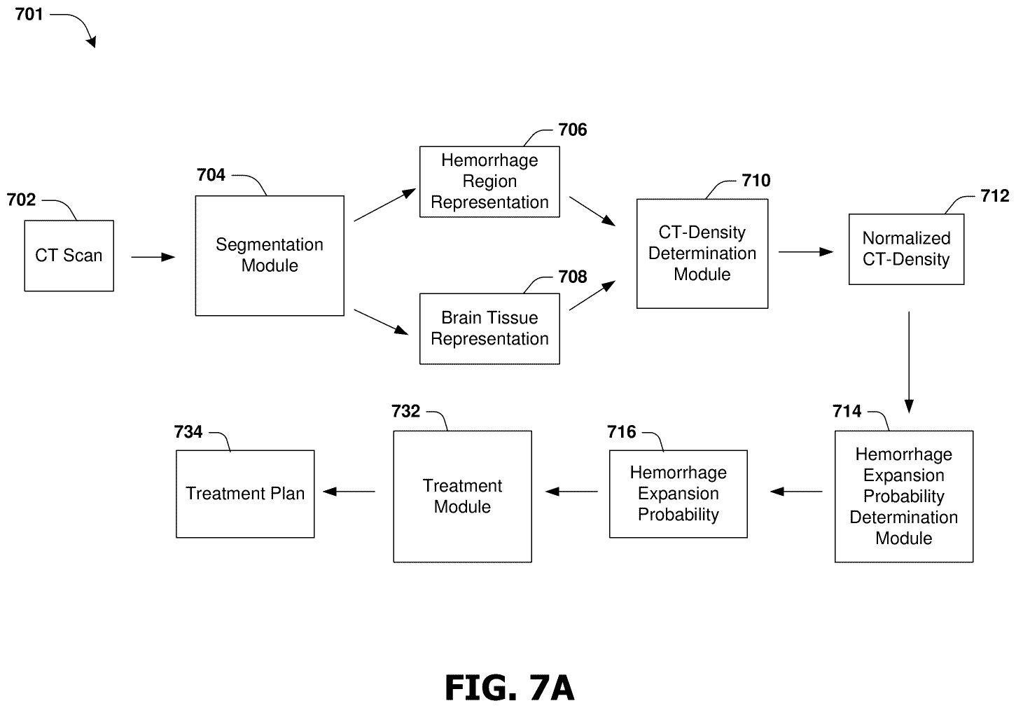

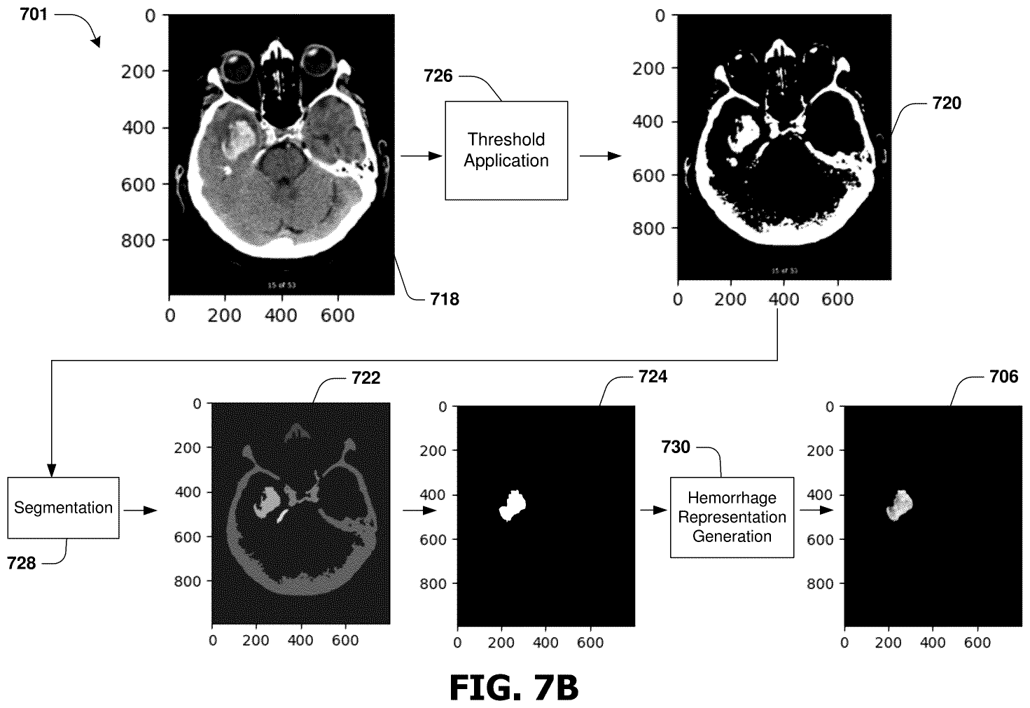

An embodiment of evaluating an image of a brain of a patient and/or determining a probability of hemorrhage expansion and/or a treatment plan for the patient (automatically and/or without manual user intervention, for example) is illustrated by an example method 600 of FIG. 6, and is further described in conjunction with system 701 of FIGS. 7A-7B. At 602 of FIG. 6, a CT scan 702 (shown in FIG. 7A) of a brain of a patient may be received. For example, the CT scan 702 may comprise cross-sectional images corresponding to various sections (e.g., slices) of the brain. In some examples, the CT scan 702 may comprise a CTH. In some examples, the CT scan 702 may comprise a non-contrast CT scan (e.g., a CTH scan without administering the contrast agent). Embodiments are contemplated in which one or more other types of images are used to determine a probability of hemorrhage expansion of the patient using techniques of the example method 600 and/or the system 701, such as MRI images and/or other types of images.

At 604 of FIG. 6, a hemorrhage region in the CT scan 702 may be identified. FIG. 7A illustrates the CT scan 702 being provided to a segmentation module 704, which may be used to generate a hemorrhage region representation 706 indicative of the hemorrhage region. In some examples, the segmentation module 704 may use a segmentation machine learning model to identify the hemorrhage region in the CT scan 702 and/or generate the hemorrhage region representation 706 indicative of the hemorrhage region. The segmentation machine learning model may be trained to identify the hemorrhage region in the CT scan 702 using segmentation training information (which may comprise the hemorrhage region segmentation training information used to train the hemorrhage region segmentation machine learning model). At 606 of FIG. 6, a brain tissue region that excludes the hemorrhage region may be identified in the CT scan 702. For example, the segmentation module 704 may generate a brain tissue region representation 708 indicative of the brain tissue region (that excludes the hemorrhage region indicated by the hemorrhage region representation 706, for example).

FIG. 7B illustrates one or more operations performed by the segmentation module 704 (shown in FIG. 7A) to identify the hemorrhage region and/or the brain tissue region (that excludes the hemorrhage region) in the CT scan 702. In some examples, the segmentation module 704 may apply, at 726, a CT-density threshold to an image 718 of the CT scan 702 to generate a mask 720 indicative of first regions (e.g., the hemorrhage region and/or surrounding skull bones) denser than the CT-density threshold. For example, applying the CT-density threshold to the image 718 may filter out one or more second regions (e.g., the brain tissue region excluding the hemorrhage region) from the first regions. In some examples, the brain tissue region representation 708 may be generated to include the one or more second regions (e.g., one or more parenchymal regions) that are filtered out of the image 718 (based upon the one or more second regions being less dense than the CT-density threshold, for example). In some examples, the CT-density threshold may be a pixel intensity threshold of about 150. Other values of the CT-density threshold are within the scope of the present disclosure.

In some examples, one or more segmentation operations 728 may be performed using the mask 720 (indicative of the first regions) to generate a mask 724 indicative of the hemorrhage region in the image 540 of the CT scan 502. The one or more segmentation operations 728 may comprise removing small isolated objects (e.g., small isolated areas of white pixels) from the mask 720 to generate a segmentation representation 722. In some examples, in addition to the hemorrhage region, a set of objects (e.g., one or more large isolated regions) remain in the segmentation representation 722, such as at least one of calvarium, tip of nasal bone, portion of sphenoid bone, calcified carotid arteries, and/or a portion of a right petrous apex (shown in various colors in FIG. 7B). In some examples, one or more first objects of the set of objects are filtered out (e.g., determined to not be the hemorrhage region) based upon one or more locations of one or more second anatomic landmarks associated with the patient and/or based upon an axis ŷₐ (which may be determined using one or more of the techniques provided herein with respect to FIG. 5E). For example, an object of the one or more first objects may be filtered out (e.g., determined to not be the hemorrhage region) based upon a determination that the object is within a threshold distance (e.g., one or more centimeters) of an anatomic landmark of the one or more second anatomic landmarks and/or the axis ŷₐ. In some examples, the hemorrhage region may be distinguished from one or more remaining objects of the set of objects (e.g., one or more objects that remain after filtering out the one or more first objects) based upon a comparison of a size of the hemorrhage region with the one or more remaining objects, such as based upon a determination that among the one or more remaining objects and the hemorrhage region, the hemorrhage region is a second largest object (since the hemorrhage region is smaller than the calvarium which may be included in the one or more remaining objects, for example).

In some examples, the one or more second anatomic landmarks may comprise a right inferior orbital rim, a left inferior orbital rim, a posterior tentorial incisura, a right lens of the patient, a left lens of the patient, a cerebellar tonsils of the patient, and/or one or more other landmarks. In some examples, the one or more second anatomic landmarks may be determined (using the CT scan 702, for example) using one or more of the techniques provided herein with respect to determining the one or more anatomic landmarks indicated by the anatomic landmark dataset 508.

In some examples, at 730, the hemorrhage region representation 706 may be generated based upon the mask 724 (e.g., binary mask) and/or the image 718 of the CT scan 702. For example, the mask 724 may be combined with (e.g., multiplied by) the image 718 of the CT scan 702 to generate the hemorrhage region representation 706. In some examples, the hemorrhage region representation 706 may comprise pixel values, of the hemorrhage region, indicated by the image 718 (and thus the hemorrhage region representation 706 may be usable to determine a CT-density associated with the hemorrhage region, for example). In some examples, one, some, or all of the techniques and/or operations provided herein with respect to FIG. 7B (e.g., techniques and/or operations provided herein with respect to acts 726, 728 and/or 730) may be implemented and/or performed by the hemorrhage region segmentation module 506 to identify the hemorrhage region in the image 540 of the CT scan 502 and/or generate the hemorrhage region representation 510 (shown in FIG. 5C).

In some examples, a plurality of hemorrhage region representations (comprising the hemorrhage region representation 706) and/or a plurality of brain tissue region representations (comprising the brain tissue region representation 708) may be generated based upon a plurality of images (comprising the image 718) of the CT scan 702. The plurality of images may comprise some or all axial slices of the CT scan 702. Hemorrhage region representations of the plurality of hemorrhage region representations may be generated using one or more of the techniques provided herein with respect to generating the hemorrhage region representation 706. Brain tissue region representations of the plurality of brain tissue region representations may be generated using one or more of the techniques provided herein with respect to generating the brain tissue region representation 708.

At 608 of FIG. 6, a normalized CT-density 712 (shown in FIG. 7A) associated with the hemorrhage region may be determined based upon the hemorrhage region (indicated by the hemorrhage region representation 706, for example) and/or the brain tissue region (indicated by the brain tissue region representation 708, for example). In some examples, the normalized CT-density 712 may be determined based upon a hemorrhage CT-density of the hemorrhage region and/or a brain tissue CT-density of the brain tissue region (that excludes the hemorrhage region).

In some examples, the hemorrhage CT-density of the hemorrhage region may be determined based upon the hemorrhage region representation 706. For example, the hemorrhage CT-density of the hemorrhage region may be determined by determining an average density of densities indicated by voxels, indicated by the hemorrhage region representation 706, inside the (segmented) hemorrhage region (e.g., voxels of the CT scan 702 may each be indicative of density in units of pixel intensity, Hounsfield Units, or other units). Alternatively and/or additionally, the hemorrhage CT-density of the hemorrhage region may be determined based upon the plurality of hemorrhage region representations. For example, the hemorrhage CT-density of the hemorrhage region may be determined to be an average density of densities indicated by voxels, of the plurality of hemorrhage region representations, inside the (segmented) hemorrhage region.

In some examples, the brain tissue CT-density of the brain tissue region may be determined based upon the brain tissue region representation 708. For example, the brain tissue CT-density of the brain tissue region may be determined by determining an average density of densities indicated by voxels, indicated by the brain tissue region representation 708, inside the (segmented) brain tissue region. Alternatively and/or additionally, the brain tissue CT-density of the brain tissue region may be determined based upon the plurality of brain tissue region representations. For example, the brain tissue CT-density of the brain tissue region may be determined to be an average density of densities indicated by voxels, of the plurality of brain tissue region representations, inside the (segmented) brain tissue region.

In some examples, the CT-density determination module 710 may combine the hemorrhage CT-density of the hemorrhage region and the brain tissue CT-density of the brain tissue region (that excludes the hemorrhage region) to determine the normalized CT-density 712. For example, the CT-density determination module 710 may determine the normalized CT-density 712 by dividing the hemorrhage CT-density by the brain tissue CT-density. In some examples, the normalized CT-density 712 may be a volumetric intracranial hemorrhage (ICH) density (e.g., volumetric bgICH density).

At 610 of FIG. 6, a hemorrhage expansion probability 716 (e.g., probability of hematoma expansion) may be determined based upon the normalized CT-density associated with the hemorrhage region. For example, the hemorrhage expansion probability 716 may be indicative of a probability that the hemorrhage region of the patient is expanding, and/or expanding at a rate of expansion that is least a threshold rate of expansion (e.g., an increase in hemorrhage volume of the hemorrhage region at a rate of 10 milliliters over six hours).

FIG. 7A illustrates the hemorrhage expansion probability 716 being determined by a hemorrhage expansion probability determination module 714 based upon the normalized CT-density 712. In some examples, a decrease of the normalized CT-density 712 may correspond to a higher value of the hemorrhage expansion probability 716. Alternatively and/or additionally, other information associated with the patient may be used by the hemorrhage expansion probability determination module 714 to determine the hemorrhage expansion probability 716, such as at least one of whether a spot sign was detected via a CTA of the patient, an age of the patient, a stroke score (e.g., a NIHSS score), an IVH extent score (e.g., a mGS score), and/or other information associated with the patient.

In some examples, the hemorrhage expansion probability 716 may be determined using a hemorrhage expansion machine learning model. In some examples, the hemorrhage expansion machine learning model may comprise a second multivariate logistic regression model. The hemorrhage expansion machine learning model may be trained to determine hemorrhage expansion probabilities for patients using hemorrhage expansion probability determination training information. For example, the hemorrhage expansion probability determination training information may comprise sets of patient information associated with a plurality of patients, and/or label information (e.g., ground truth information) that is indicative of sets of hemorrhage expansion information associated with the plurality of patients.

A set of patient information (indicated by the hemorrhage expansion probability determination training information) associated with a second patient may be indicative of a second normalized CT-density associated with the second patient (which may be determined using one or more of the techniques provided herein with respect to determining the normalized CT-density 712), whether a spot sign was detected via a CTA of the second patient, an age of the second patient, a stroke score (e.g., a NIHSS score) associated with the second patient, an IVH extent score (e.g., a mGS score) associated with the second patient, and/or other information associated with the second patient. A set of hemorrhage expansion information (indicated by the label information of the hemorrhage expansion probability determination training information) associated with the second patient may be indicative of whether the second patient had hemorrhage expansion (e.g., whether a second hemorrhage region of the second patient is expanding and/or expanding at a rate of expansion that is least the threshold rate of expansion), and/or other information associated with the second patient.

In some examples, a treatment module 732 (shown in FIG. 7A) may generate a treatment plan 734 for the patient based upon the hemorrhage expansion probability 716. For example, the treatment plan 734 may be indicative of treating the patient with the first type of treatment (e.g., surgical hemorrhage evacuation) based upon the hemorrhage expansion probability being greater than a first hemorrhage expansion probability threshold. Alternatively and/or additionally, the treatment plan 526 may be generated to be indicative of not treating the patient with the first type of treatment (e.g., surgical hemorrhage evacuation) based upon the hemorrhage expansion probability being less than the first hemorrhage expansion probability threshold. In some examples, the treatment plan 526 may be generated to indicate treating the patient with the second type of treatment (e.g., non-surgical medical treatment) based upon the hemorrhage expansion probability being less than the first hemorrhage expansion probability threshold.

In some examples, the treatment plan 734 may be provided to a client device for display. The client device (and/or the treatment plan 734 displayed on the client device) may be accessible to at least one of the patient, a healthcare professional (e.g., physician, surgeon, nurse, etc.) associated with the patient, etc. In some examples, one or more treatments (e.g., the first type of treatment and/or the second type of treatment) indicated by the treatment plan 734 may be performed automatically (and/or without user intervention) and/or with supervision and/or guidance of the healthcare professional. Alternatively and/or additionally, the treatment plan 734 may comprise a guide for performing the one or more treatments, which may be used by the healthcare professional (and/or a healthcare machine and/or robot) to perform the one or more treatments.

In some examples, the one or more treatments may comprise a surgical hemorrhage evacuation (e.g., the first type of treatment). For example, the surgical hemorrhage evacuation may be scheduled for the patient and/or performed on the patient in response to the treatment plan 734 indicating the surgical hemorrhage evacuation. In some examples, the surgical hemorrhage evacuation (which may also be referred to as surgical hematoma evacuation) may comprise MIS for hemorrhage evacuation.

In some examples, the one or more treatments may comprise a non-surgical medical treatment (e.g., the second type of treatment). For example, the non-surgical medical treatment may be administered to the patient in response to the treatment plan 734 indicating the non-surgical medical treatment. In some examples, the non-surgical medical treatment may comprise a blood pressure control treatment, an anticoagulation reversal treatment to reverse anticoagulation (if present, for example), an intracranial pressure management treatment, and/or providing one or more supportive care treatments.

In some examples, the hemorrhage expansion probability 716 may be determined using the normalized CT-density 712 with similar accuracy, but with reduced cost and/or time spent, in comparison with determining the hemorrhage expansion probability 716 by generating a CTA of the patient (which may require administering contrast dye into the patient's blood vessels, for example) and analyzing the CTA to determine whether a spot sign is apparent in the CTA. For example, the normalized CT-density 712 may be determined using a CTH scan performed without administering the contrast agent, which may enable the hemorrhage expansion probability 716 to be determined more quickly and/or in a less expensive manner.

In some examples, a CTA of the brain of the patient may be received. For example, a CTA procedure may be scheduled for the patient and/or the CTA may be generated via the CTA procedure in response to the hemorrhage expansion probability 716 being greater than a second hemorrhage expansion probability threshold and/or a confidence score of the hemorrhage expansion probability 716 being less than a confidence score threshold. In some examples, the CTA may be analyzed to determine whether the CTA is indicative of a spot sign (e.g., a radiographic marker). In some examples, a second hemorrhage expansion probability may be determined (using the hemorrhage expansion machine learning model, for example) based upon the normalized CT-density 712 and/or a determination of whether the CTA is indicative of the spot sign (and/or a representation of the spot sign detected in the CTA). In some examples, the treatment plan 734 may be determined based upon the second hemorrhage expansion probability. Embodiments are contemplated in which the hemorrhage expansion probability 716 is determined (using the hemorrhage expansion machine learning model, for example) based upon the normalized CT-density 712 and whether the CTA is indicative of the spot sign (and/or a representation of the spot sign detected in the CTA).

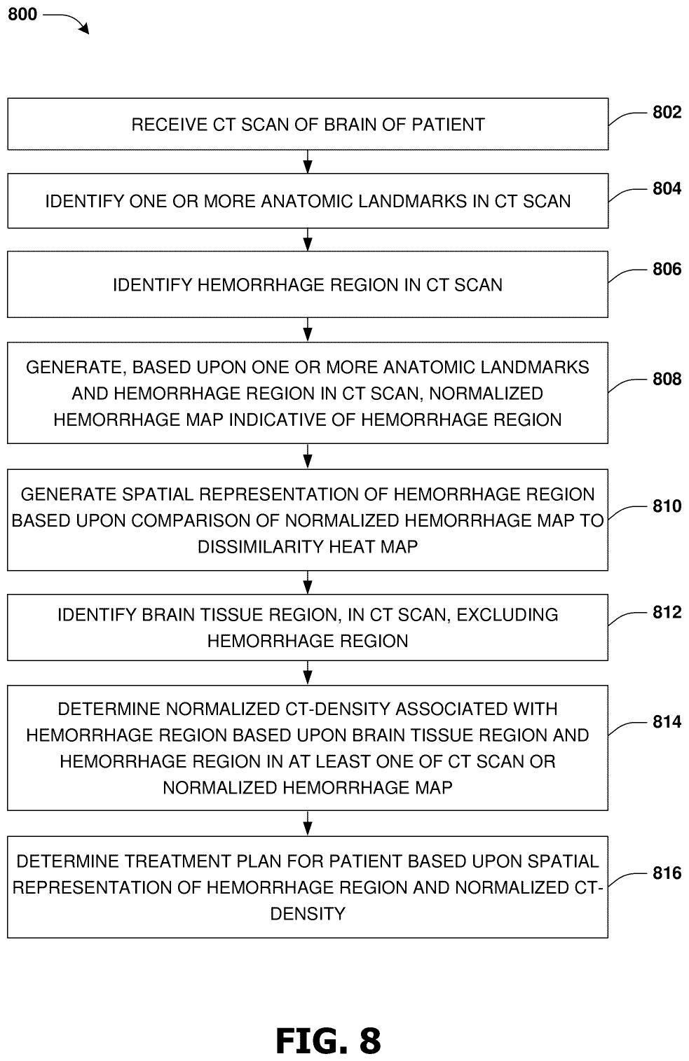

An embodiment of determining a treatment plan for a patient is illustrated by an example method 800 of FIG. 8. At 802, a computed CT scan (e.g., the CT scan 502 and/or the CT scan 702) of a brain of a patient may be received. At 804, one or more anatomic landmarks (e.g., the one or more anatomic landmarks indicated by the anatomic landmark dataset 508 and/or the one or more second anatomic landmarks) in the CT scan may be identified. At 806, a hemorrhage region in the CT scan may be identified. For example, the hemorrhage region may comprise an ICH region, a bgICH region, and/or other type of hemorrhage region. At 808, a normalized hemorrhage map (e.g., the normalized hemorrhage map 514) indicative of the hemorrhage region may be generated based upon the one or more anatomic landmarks and/or the hemorrhage region in the CT scan. At 810, a spatial representation (e.g., the spatial representation 522) of the hemorrhage region may be generated based upon a comparison of the normalized hemorrhage map to a dissimilarity heat map (e.g., the dissimilarity heat map 520). At 812, a brain tissue region, in the CT scan, excluding the hemorrhage region may be identified (e.g., the brain tissue region indicated by the brain tissue region representation 708). At 814, a normalized CT-density associated with the hemorrhage region may be determined based upon the brain tissue region and the hemorrhage region in at least one of the CT scan or the normalized hemorrhage map. At 816, a treatment plan (e.g., comprising treatment information provided herein with respect to the treatment plan 526 and/or treatment information provided herein with respect to the treatment plan 734) for the patient may be generated based upon the spatial representation of the hemorrhage region and the normalized CT-density. In some examples, one or more treatments (e.g., at least one of surgical hemorrhage evacuation, non-surgical medical treatment, etc.) may be performed based upon the treatment plan.

In some examples, a functional outcome (e.g., the first functional outcome) associated with the patient may be determined based upon the spatial representation. A probability of hemorrhage expansion (e.g., the hemorrhage expansion probability 716) may be determined based upon the CT-density of the hemorrhage region. The treatment plan may be determined based upon the functional outcome and the probability of hemorrhage expansion (which may provide for increased accuracy of the treatment plan).

In some examples, each machine learning model of one, some and/or all machine learning models of the present disclosure (e.g., the landmark identification machine learning model, the hemorrhage region segmentation machine learning model, the functional outcome machine learning model, the segmentation machine learning model, the hemorrhage expansion machine learning model, etc.) may comprise at least one of a neural network, such as a convolutional neural network, a tree-based model, a machine learning model used to perform linear regression, a machine learning model used to perform logistic regression, a decision tree model, a support vector machine (SVM), a Bayesian network model, a k-Nearest Neighbors (k-NN) model, a K-Means model, a random forest model, a machine learning model used to perform dimensional reduction, a machine learning model used to perform gradient boosting, etc.

According to some embodiments, a method is provided. The method includes receiving one or more images of a brain of a patient; identifying one or more anatomic landmarks in the one or more images; identifying a hemorrhage region in the one or more images; generating, based upon the one or more anatomic landmarks and the hemorrhage region in the one or more images, a normalized hemorrhage map indicative of the hemorrhage region; generating a spatial representation of the hemorrhage region based upon a comparison of the normalized hemorrhage map to a dissimilarity heat map; and determining a treatment plan for the patient based upon the spatial representation of the hemorrhage region.

According to some embodiments, generating the normalized hemorrhage map includes mapping, based upon the one or more anatomic landmarks, coordinates of the hemorrhage region in the one or more images to coordinates of a stereotactic reference frame.

According to some embodiments, the one or more images include one or more images of a computed tomography (CT) scan.

According to some embodiments, the method includes identifying the one or more anatomic landmarks includes identifying a left inferior orbital rim; a right inferior orbital rim; and/or a posterior tentorial incisura.

According to some embodiments, the method includes generating the dissimilarity heat map based upon a comparison of a first set of hemorrhage maps associated with a first group of patients to a second set of hemorrhage maps associated with a second group of patients.

According to some embodiments, each map of the first set of hemorrhage maps is indicative of a basal ganglia Intracranial Hemorrhage (bgICH) spatial distribution of a patient associated with one or more first functional outcomes; and/or each map of the second set of hemorrhage maps is indicative of a bgICH spatial distribution of a patient associated with one or more second functional outcomes.

According to some embodiments, the method includes performing modified Rankin Scale (mRS)-based categorization to determine the first set of hemorrhage maps and the second set of hemorrhage maps.

According to some embodiments, performing the mRS-based categorization includes including a first hemorrhage map in the first set of hemorrhage maps based upon a first mRS score associated with the first hemorrhage map meeting a threshold mRS score; and including a second hemorrhage map in the second set of hemorrhage maps based upon a second mRS score associated with the second hemorrhage map not meeting the threshold mRS score.

According to some embodiments, the one or more anatomic landmarks are identified utilizing a first machine learning model; and/or the hemorrhage region is identified utilizing a second machine learning model.

According to some embodiments, the method includes determining a functional outcome associated with the patient based upon the spatial representation, wherein determining the treatment plan includes determining the treatment plan based upon the functional outcome.

According to some embodiments, determining the treatment plan includes determining the treatment plan to include a surgical hemorrhage evacuation, and the method includes performing the surgical hemorrhage evacuation.

According to some embodiments, determining the treatment plan includes determining the treatment plan to include a non-surgical medical treatment, and the method includes administering the non-surgical medical treatment to the patient.

According to some embodiments, a computing device is provided. The computing device includes a processor and memory including processor-executable instructions that when executed by the processor cause performance of operations including receiving a computed tomography (CT) scan of a brain of a patient; identifying a hemorrhage region in the CT scan; identifying a brain tissue region, in the CT scan, excluding the hemorrhage region; determining a normalized CT-density associated with the hemorrhage region based upon the hemorrhage region and the brain tissue region; and determining a probability of hemorrhage expansion based upon the normalized CT-density associated with the hemorrhage region.

According to some embodiments, identifying the hemorrhage region includes applying a CT-density threshold to an image of the CT scan to generate a first mask indicative of regions denser than the CT-density threshold; segmenting the first mask to identify a region, of the first mask, corresponding to the hemorrhage region; and mapping the region of the first mask to the image of the CT scan.

According to some embodiments, identifying the hemorrhage region includes applying a CT-density threshold to an image of the CT scan to generate a first mask indicative of regions denser than the CT-density threshold; and comparing the first mask with the image of the CT scan to identify the brain tissue region.

According to some embodiments, determining the normalized CT-density includes determining a hemorrhage CT-density of the hemorrhage region; determining a brain tissue CT-density of the brain tissue region; and determining the normalized CT-density based upon the hemorrhage CT-density and the brain tissue CT-density.

According to some embodiments, the operations include receiving a computed tomography angiogram (CTA) of the brain; and analyzing the CTA to determine whether the CTA is indicative of a spot sign, wherein determining the probability of hemorrhage expansion is based upon the normalized CT-density and whether the CTA is indicative of the spot sign.

According to some embodiments, the operations include determining a treatment plan for the patient based upon the probability of hemorrhage expansion.

According to some embodiments, a non-transitory machine readable medium is provided. The non-transitory machine readable medium has stored thereon processor-executable instructions that when executed cause performance of operations including receiving a computed tomography (CT) scan of a brain of a patient; identifying one or more anatomic landmarks in the CT scan; identifying a hemorrhage region in the CT scan; generating, based upon the one or more anatomic landmarks and the hemorrhage region in the CT scan, a normalized hemorrhage map indicative of the hemorrhage region; generating a spatial representation of the hemorrhage region based upon a comparison of the normalized hemorrhage map to a dissimilarity heat map; identifying a brain tissue region, in the CT scan, excluding the hemorrhage region; determining a normalized CT-density associated with the hemorrhage region based upon the brain tissue region and the hemorrhage region in at least one of the CT scan or the normalized hemorrhage map; and determining a treatment plan for the patient based upon the spatial representation of the hemorrhage region and the normalized CT-density.

According to some embodiments, the operations include determining a functional outcome associated with the patient based upon the spatial representation; and determining a probability of hemorrhage expansion based upon the CT-density of the hemorrhage region, wherein determining the treatment plan includes determining the treatment plan based upon the functional outcome and the probability of hemorrhage expansion.

According to some embodiments, a method is provided which includes at least one aspect as described in the present disclosure and/or shown in the figures.

According to some embodiments, a method is provided which includes plural aspects as described in the present disclosure and/or shown in the figures.

According to some embodiments, a system is provided which includes at least one aspect as described in the present disclosure and/or shown in the figures.

According to some embodiments, a system is provided which includes plural aspects as described in the present disclosure and/or shown in the figures.

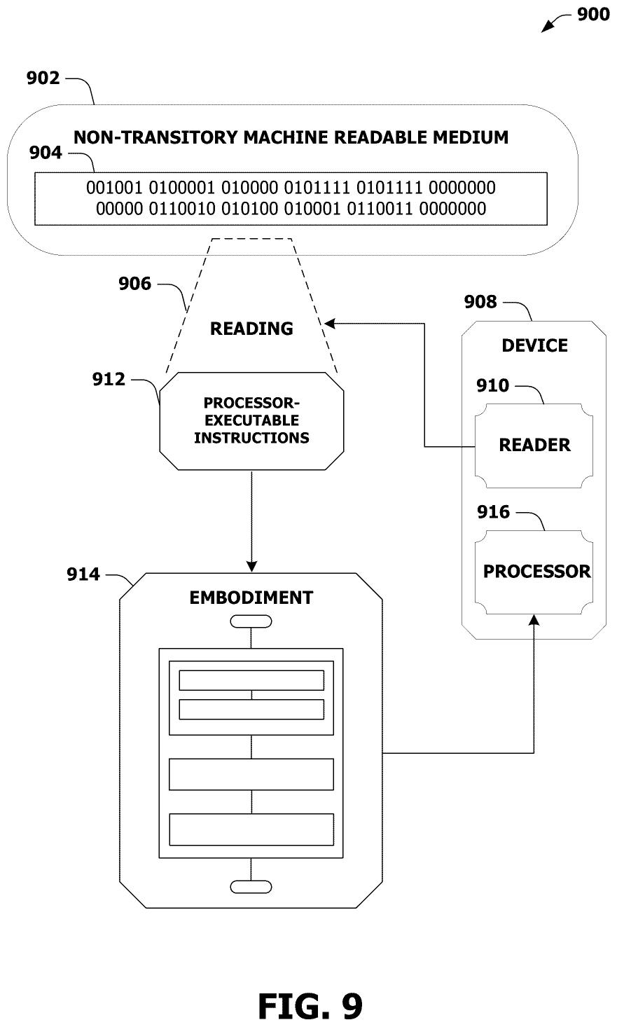

FIG. 9 is an illustration of a scenario 900 involving an example non-transitory machine readable medium 902. The non-transitory machine readable medium 902 may comprise processor-executable instructions 912 that when executed by a processor 916 cause performance (e.g., by the processor 916) of at least some of the provisions herein (e.g., embodiment 914).

The non-transitory machine readable medium 902 may comprise a memory semiconductor (e.g., a semiconductor utilizing static random access memory (SRAM), dynamic random access memory (DRAM), and/or synchronous dynamic random access memory (SDRAM) technologies), a platter of a hard disk drive, a flash memory device, or a magnetic or optical disc (such as a compact disc (CD), digital versatile disc (DVD), or floppy disk).

The example non-transitory machine readable medium 902 stores computer-readable data 904 that, when subjected to reading 906 by a reader 910 of a device 908 (e.g., a read head of a hard disk drive, or a read operation invoked on a solid-state storage device), express the processor-executable instructions 912.

In some embodiments, the processor-executable instructions 912, when executed, cause performance of operations, such as at least some of the example method 400 of FIG. 4, at least some of the example method 600 of FIG. 6 and/or at least some of the example method 800 of FIG. 8, for example. In some embodiments, the processor-executable instructions 912 are configured to cause implementation of a system, such as at least some of the example system 501 of FIGS. 5A-5G, and/or at least some of the example system 701 of FIGS. 7A-7B, for example.

As used in this application, “component,” “module,” “system”, “interface”, and/or the like are generally intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers.

Unless specified otherwise, “first,” “second,” and/or the like are not intended to imply a temporal aspect, a spatial aspect, an ordering, etc. Rather, such terms are merely used as identifiers, names, etc. for features, elements, items, etc. For example, a first object and a second object generally correspond to object A and object B or two different or two identical objects or the same object.

Moreover, “example” is used herein to mean serving as an instance, illustration, etc., and not necessarily as advantageous. As used herein, “or” is intended to mean an inclusive “or” rather than an exclusive “or”. In addition, “a” and “an” as used in this application are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, and/or variants thereof are used in either the detailed description or the claims, such terms are intended to be inclusive in a manner similar to the term “comprising”.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing at least some of the claims.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, many modifications may be made to this configuration without departing from the scope or spirit of the claimed subject matter.

Various operations of embodiments are provided herein. In an embodiment, one or more of the operations described may constitute computer readable instructions stored on one or more computer and/or machine readable media, which if executed will cause the operations to be performed. The order in which some or all of the operations are described should not be construed as to imply that these operations are necessarily order dependent. Alternative ordering will be appreciated by one skilled in the art having the benefit of this description. Further, it will be understood that not all operations are necessarily present in each embodiment provided herein. Also, it will be understood that not all operations are necessary in some embodiments.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

What is claimed is:

1. A method comprising:
receiving one or more images of a brain of a patient;
identifying one or more anatomic landmarks in the one or more images;
identifying a hemorrhage region in the one or more images;
generating, based upon the one or more anatomic landmarks and the hemorrhage region in the one or more images, a normalized hemorrhage map indicative of the hemorrhage region;
generating a spatial representation of the hemorrhage region based upon a comparison of the normalized hemorrhage map to a dissimilarity heat map; and
determining a treatment plan for the patient based upon the spatial representation of the hemorrhage region.

2. The method of claim 1, wherein generating the normalized hemorrhage map comprises:
mapping, based upon the one or more anatomic landmarks, coordinates of the hemorrhage region in the one or more images to coordinates of a stereotactic reference frame.

3. The method of claim 1, wherein:
the one or more images comprise one or more images of a computed tomography (CT) scan.

4. The method of claim 1, wherein identifying the one or more anatomic landmarks comprises identifying at least one of:
a left inferior orbital rim;
a right inferior orbital rim; or
a posterior tentorial incisura.

5. The method of claim 1, comprising:
generating the dissimilarity heat map based upon a comparison of a first set of hemorrhage maps associated with a first group of patients to a second set of hemorrhage maps associated with a second group of patients.

6. The method of claim 5, wherein at least one of:
each map of the first set of hemorrhage maps is indicative of a basal ganglia Intracranial Hemorrhage (bgICH) spatial distribution of a patient associated with one or more first functional outcomes; or
each map of the second set of hemorrhage maps is indicative of a bgICH spatial distribution of a patient associated with one or more second functional outcomes.

7. The method of claim 5, comprising:
performing modified Rankin Scale (mRS)-based categorization to determine the first set of hemorrhage maps and the second set of hemorrhage maps.

8. The method of claim 7, wherein performing the mRS-based categorization comprises:
including a first hemorrhage map in the first set of hemorrhage maps based upon a first mRS score associated with the first hemorrhage map meeting a threshold mRS score; and
including a second hemorrhage map in the second set of hemorrhage maps based upon a second mRS score associated with the second hemorrhage map not meeting the threshold mRS score.

9. The method of claim 1, wherein at least one of:
the one or more anatomic landmarks are identified utilizing a first machine learning model; or
the hemorrhage region is identified utilizing a second machine learning model.

10. The method of claim 1, comprising:
determining a functional outcome associated with the patient based upon the spatial representation, wherein determining the treatment plan comprises determining the treatment plan based upon the functional outcome.

11. The method of claim 1, wherein determining the treatment plan comprises determining the treatment plan to comprise a surgical hemorrhage evacuation, the method comprising:
performing the surgical hemorrhage evacuation.

12. The method of claim 1, wherein determining the treatment plan comprises determining the treatment plan to comprise a non-surgical medical treatment, the method comprising:
administering the non-surgical medical treatment to the patient.

13. A computing device comprising:
a processor; and
memory comprising processor-executable instructions that when executed by the processor cause performance of operations comprising:
receiving a computed tomography (CT) scan of a brain of a patient;
identifying a hemorrhage region in the CT scan;
identifying a brain tissue region, in the CT scan, excluding the hemorrhage region;
determining a normalized CT-density associated with the hemorrhage region based upon the hemorrhage region and the brain tissue region; and
determining a probability of hemorrhage expansion based upon the normalized CT-density associated with the hemorrhage region.

14. The computing device of claim 13, wherein identifying the hemorrhage region comprises:
applying a CT-density threshold to an image of the CT scan to generate a first mask indicative of regions denser than the CT-density threshold;
segmenting the first mask to identify a region, of the first mask, corresponding to the hemorrhage region; and
mapping the region of the first mask to the image of the CT scan.

15. The computing device of claim 13, wherein identifying the hemorrhage region comprises:
applying a CT-density threshold to an image of the CT scan to generate a first mask indicative of regions denser than the CT-density threshold; and
comparing the first mask with the image of the CT scan to identify the brain tissue region.

16. The computing device of claim 13, wherein determining the normalized CT-density comprises:
determining a hemorrhage CT-density of the hemorrhage region;
determining a brain tissue CT-density of the brain tissue region; and
determining the normalized CT-density based upon the hemorrhage CT-density and the brain tissue CT-density.

17. The computing device of claim 13, the operations comprising:
receiving a computed tomography angiogram (CTA) of the brain; and
analyzing the CTA to determine whether the CTA is indicative of a spot sign, wherein determining the probability of hemorrhage expansion is based upon the normalized CT-density and whether the CTA is indicative of the spot sign.

18. The computing device of claim 13, the operations comprising:
determining a treatment plan for the patient based upon the probability of hemorrhage expansion.

19. A non-transitory machine readable medium having stored thereon processor-executable instructions that when executed cause performance of operations comprising:
receiving a computed tomography (CT) scan of a brain of a patient;
identifying one or more anatomic landmarks in the CT scan;
identifying a hemorrhage region in the CT scan;
generating, based upon the one or more anatomic landmarks and the hemorrhage region in the CT scan, a normalized hemorrhage map indicative of the hemorrhage region;
generating a spatial representation of the hemorrhage region based upon a comparison of the normalized hemorrhage map to a dissimilarity heat map;
identifying a brain tissue region, in the CT scan, excluding the hemorrhage region;
determining a normalized CT-density associated with the hemorrhage region based upon the brain tissue region and the hemorrhage region in at least one of the CT scan or the normalized hemorrhage map; and
determining a treatment plan for the patient based upon the spatial representation of the hemorrhage region and the normalized CT-density.

20. The non-transitory machine readable medium of claim 19, the operations comprising:
determining a functional outcome associated with the patient based upon the spatial representation; and
determining a probability of hemorrhage expansion based upon the CT-density of the hemorrhage region, wherein determining the treatment plan comprises determining the treatment plan based upon the functional outcome and the probability of hemorrhage expansion.
